(19) United States
(12) Patent Application Publication
(10) Pub. No.: US 2025/0302798 A1
(43) Pub. Date: Oct. 2, 2025

(54) STING INHIBITORS AND USE THEREOF

(71) Applicant: CARMEL HAIFA UNIVERSITY ECONOMIC CORPORATION LTD., Haifa (IL)

(72) Inventors: Amiram ARIEL, Kiryat Motzkin (IL); Nofar BEN JASHAR, Lod (IL); Sagie SCHIF, Haifa (IL); Uzma SAQIB, Indore (IN)

(21) Appl. No.: 18/863,244

(22) PCT Filed: Apr. 25, 2023
(86) PCT No.: PCT/IL2023/050427

§ 371 Date: Nov. 5, 2024

Related U.S. Application Data
(60) Provisional application No. 63/342,099, filed on May 14, 2022.

(51) Int. Cl. A61K 31/36; A61K 31/4155; A61K 31/4196; A61K 31/4245; A61K 31/444; A61K 31/445; A61K 31/454; A61K 31/4709; A61K 31/496; A61K 31/501; A61K 31/502; A61K 31/553; A61P 1/16

(52) U.S. Cl. CPC A61K 31/36; A61K 31/4155; A61K 31/4196; A61K 31/4245; A61K 31/444; A61K 31/445; A61K 31/454; A61K 31/4709; A61K 31/496; A61K 31/501; A61K 31/502; A61K 31/553; A61P 1/16

(57) ABSTRACT

The present invention provides novel stimulator of IFN genes inhibitors and methods of using same, such as for treating inflammatory diseases.

### CROSS-REFERENCE TO RELATED APPLICATIONS

This application claims the benefit of priority of U.S. Provisional Patent Application No. 63/342,099, titled “STING INHIBITORS AND USE THEREOF”, filed 14 May 2022, the contents of which are incorporated herein by reference in their entirety.

### FIELD OF INVENTION

The present invention relates to stimulator of IFN genes inhibitors, methods for using same, such as for treating autoimmune diseases, fibrotic diseases, and inflammatory diseases.

### BACKGROUND

By the early 1950s, it was well established that under certain conditions virus-infected cells are resistant to a second virus infection. Therefore, by some mechanism, viruses interfere with each other.

In 1957, the term “interferon” was coined by Isaacs & Lindenmann to describe a substance, likely produced by cells, that interferes with influenza infection. IFN was later shown to be a small protein, produced, and secreted by cells (reviewed in 8) following cellular detection of pathogen-associated molecular patterns, commonly known as PAMPs, by pattern-recognition receptors.

The stimulator of IFN genes (STING) protein works as both a direct cytosolic DNA sensor (CDS) and an adaptor protein in Type I interferon signaling through different molecular mechanisms. It has been shown to activate downstream transcription factors STAT6 and IRF3 through TBK1. These effectors are responsible for antiviral and innate immune responses against intracellular pathogen.

Generally, STING is an adaptor signaling component that regulates immune responses to cytosolic dsDNA derived from DNA viruses, bacteria, and cancer cells as well as in autoimmune disorders like systemic Lupus Erythematosus. Several pathologies like Aicardi Goutières syndrome (AGS), STING-associated vasculopathy with onset in infancy (SAVI), and some forms of lupus have been shown to develop due to STING activating mutations or deficiencies in signaling elements that limit STING activity. Recently, STING activation was also implicated in neurodegenerative disorders, like Parkinson's disease, Huntington's disease and Amyotrophic Lateral Sclerosis (ALS) (Decout et al Nat Rev Immunol 2021).

Whilst the STING-mediated dsDNA-sensing mechanism is critical for successful cellular protection against infections and disease progression, dysregulated STING activity leads to the excessive production of inflammatory mediators with potentially detrimental effects on surrounding cells and tissues.

While STING mediates the production of inflammatory cytokines, like TNF-α, IL-6, and IL-12, it also promotes the production of paramount anti-inflammatory cytokines, like IL-10, through the production of IFN-β.

Therefore, discovering novel compounds that regulate STING activity is a major therapeutic target, with STING antagonists/biased agonists perceived to be beneficial in autoimmune, fibrotic, and chronic inflammatory disorders and STING-mediated pathologies.

### SUMMARY

According to a first aspect, there is provided a pharmaceutical composition, comprising a pharmaceutically acceptable carrier and at least one stimulator of IFN genes (STING) inhibitor selected from the group consisting of:

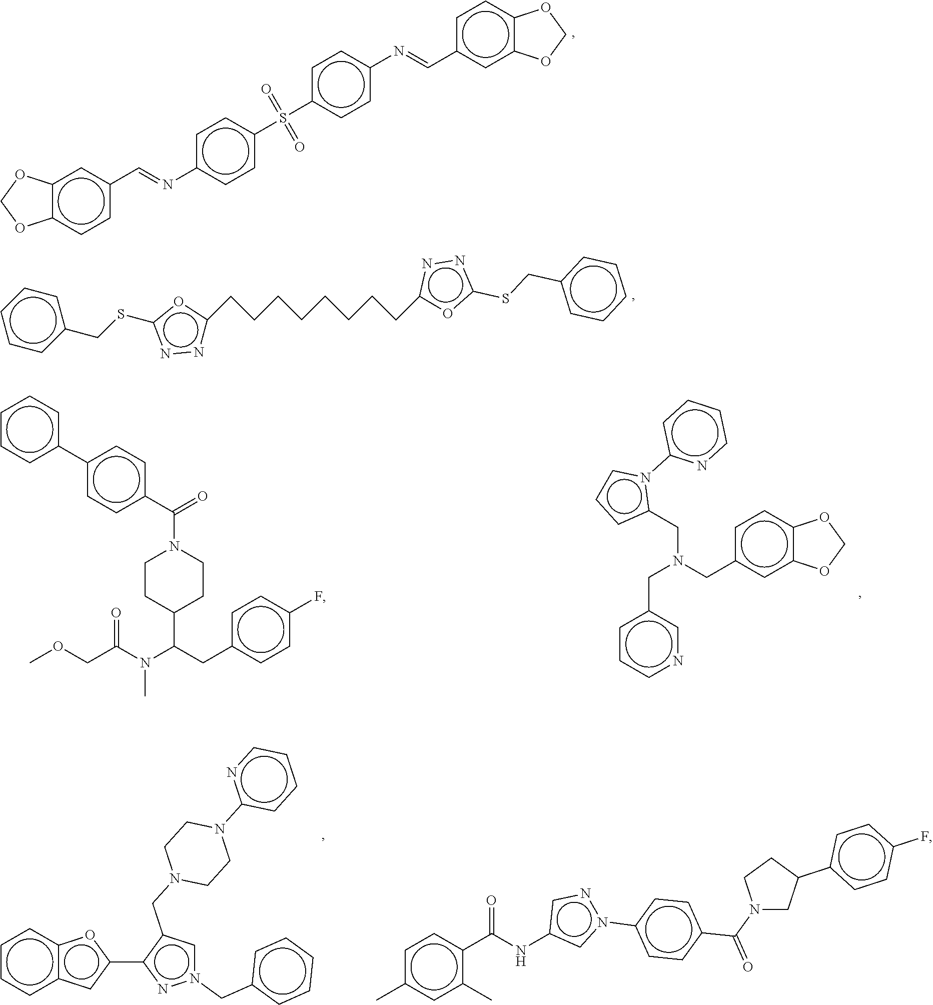

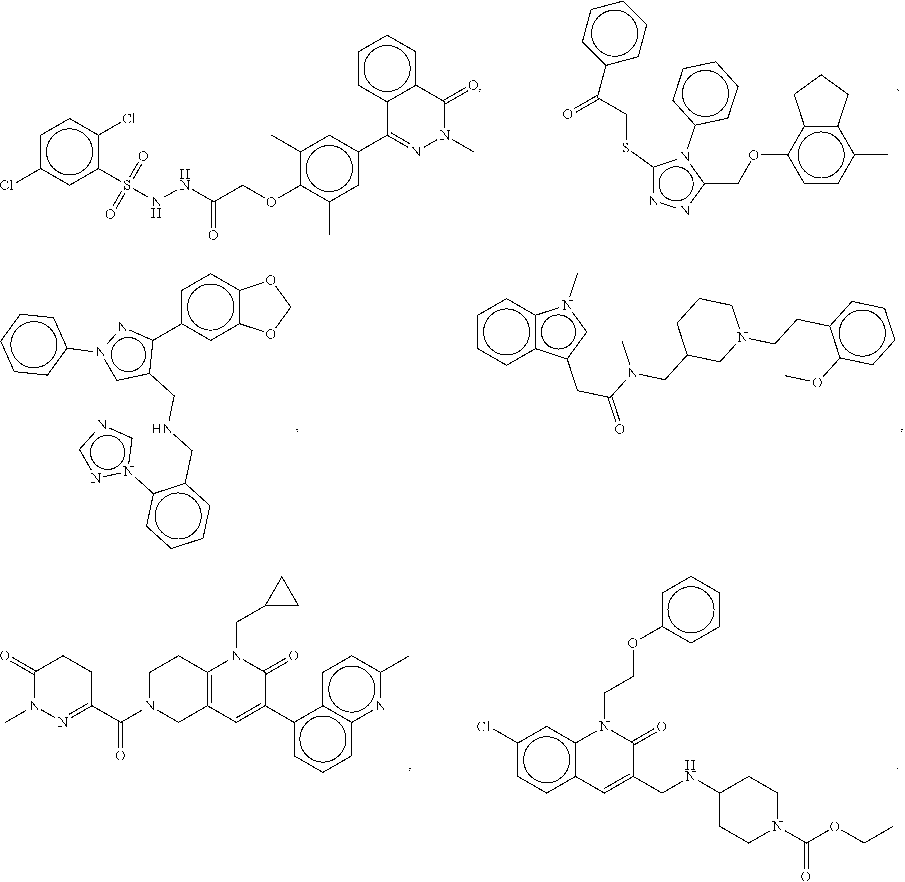

According to another aspect, there is provided method for inhibiting TNF-alpha secretion from a cell, comprising the step of contacting the cell with an effective amount of a compound selected from the group consisting of:

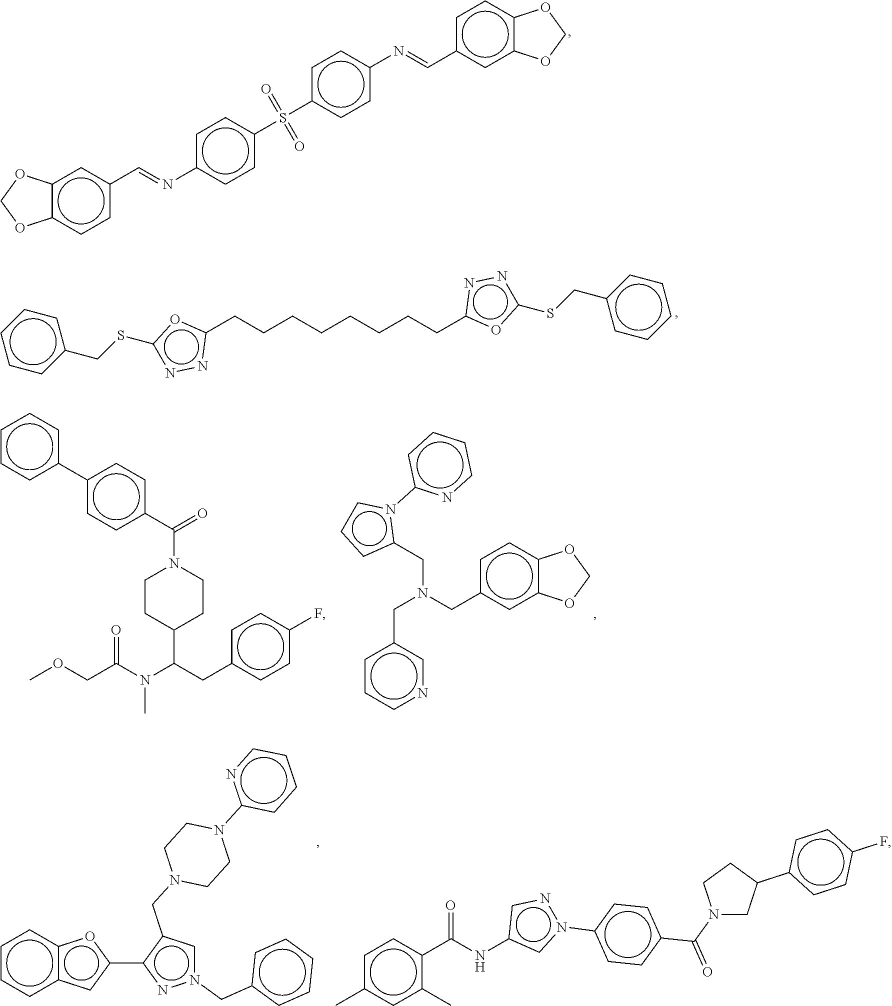

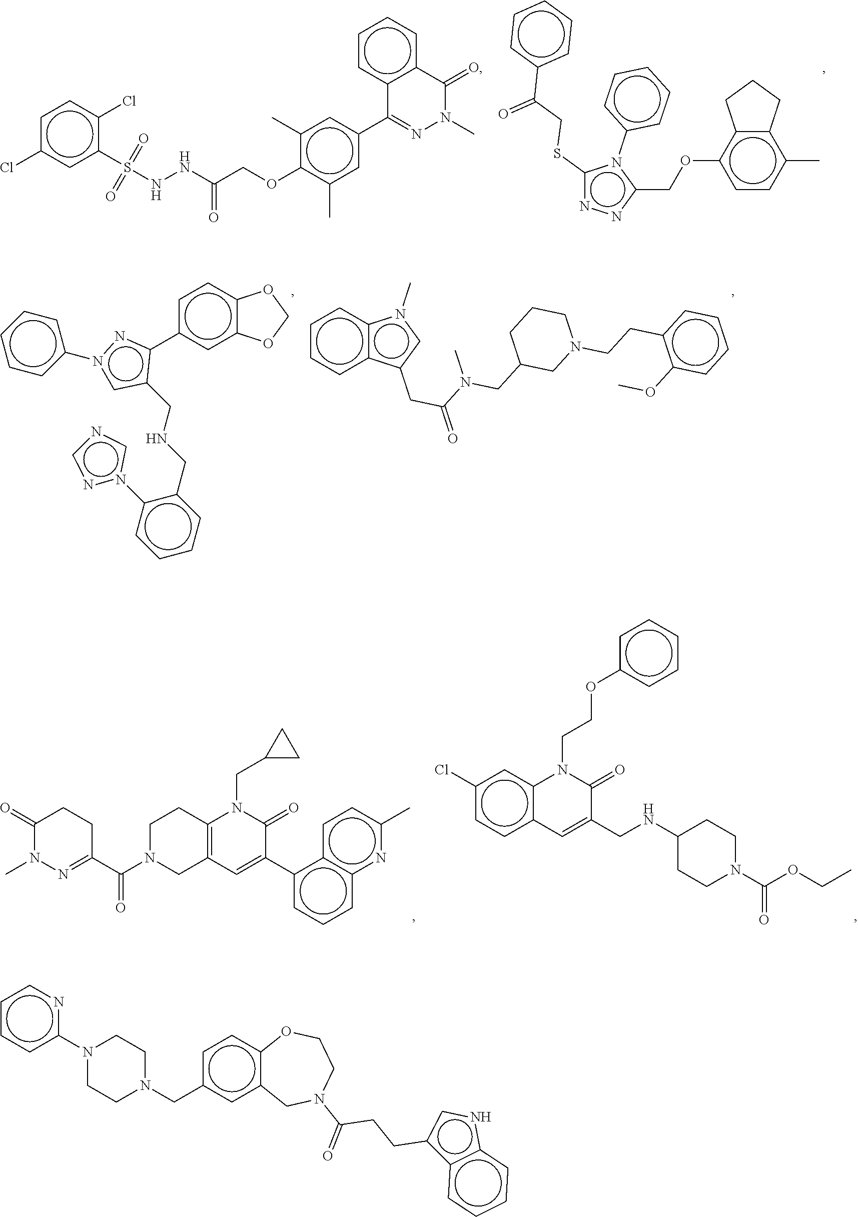

and any combination thereof, thereby inhibiting TNF-alpha secretion from said cell.

According to another aspect, there is provided a method for inhibiting STING in a cell, comprising the step of contacting the cell with an effective amount of a compound selected from the group consisting of:

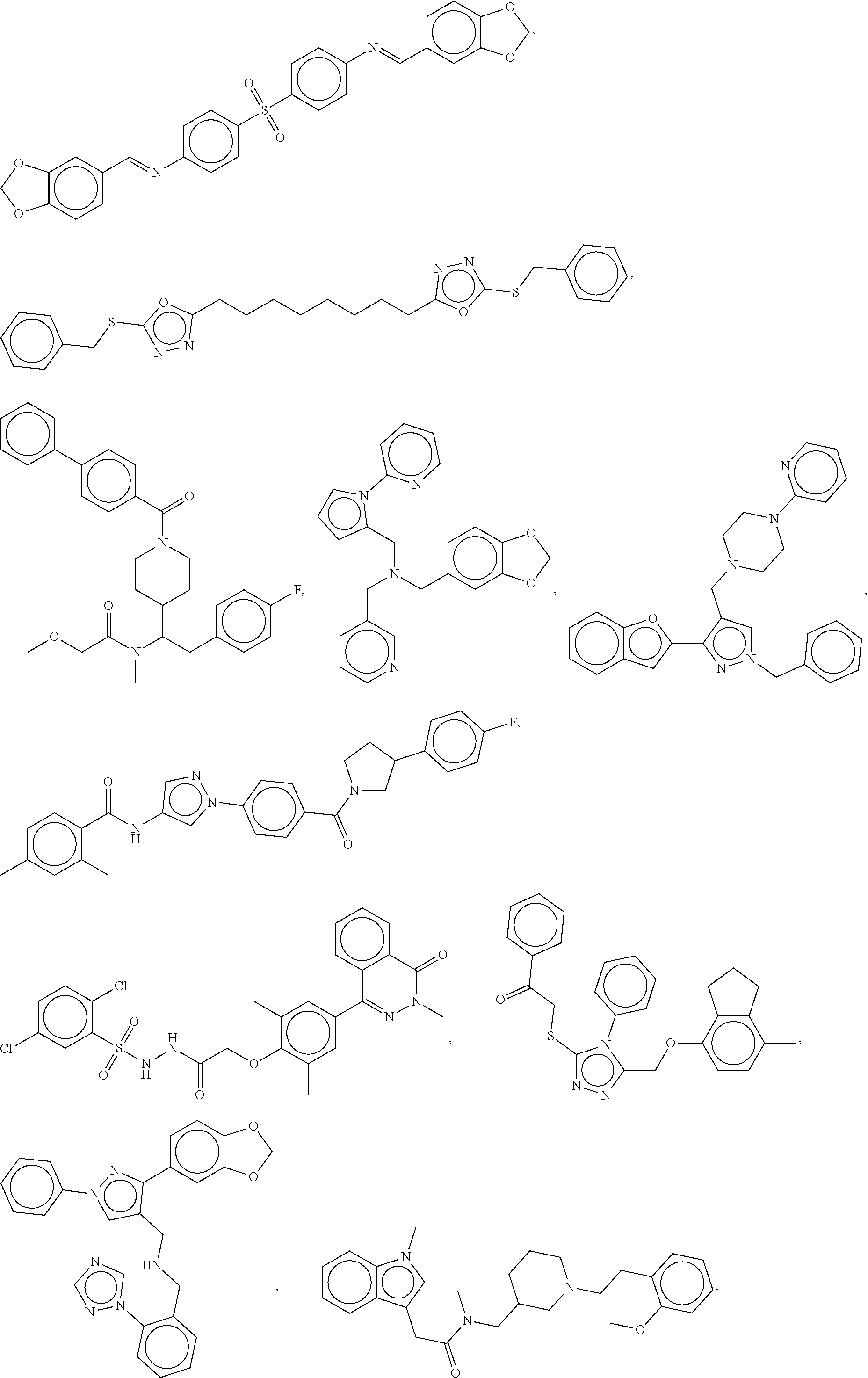

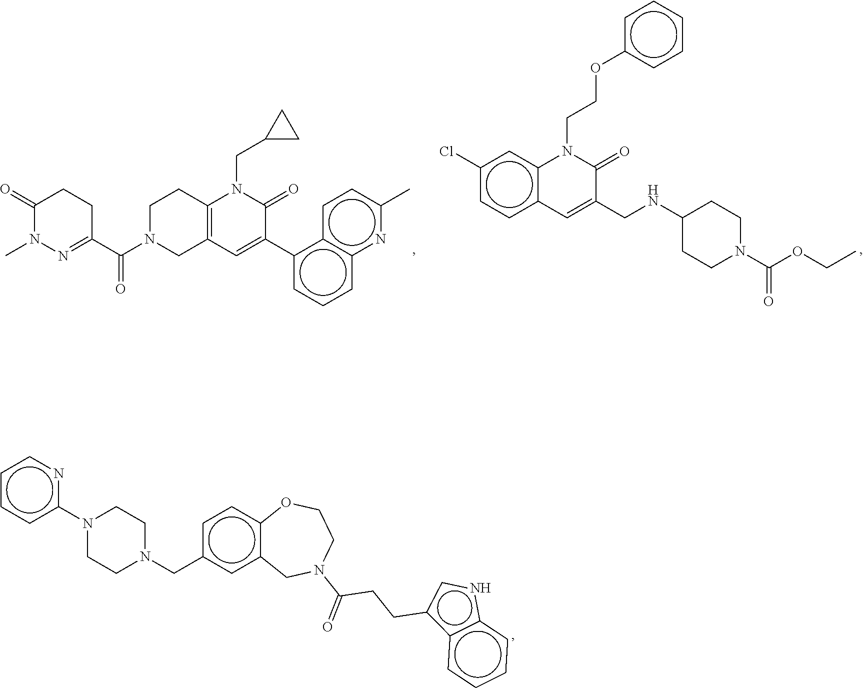

and any combination thereof, thereby inhibiting said STING in said cell.

According to another aspect, there is provided a method for treating a fibrotic disease in a subject in need thereof, the method comprising administering to the subject a therapeutically effective amount of the pharmaceutical composition disclosed herein, thereby treating a fibrotic disease in the subject.

In some embodiments, the compound is:

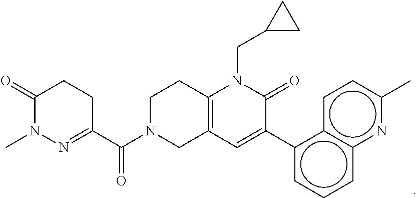

In some embodiments, the compound induces IL-10 secretion, IFN-beta secretion, or both, from the cell.

In some embodiments, the compound inhibits IL-10 secretion, IFN-beta secretion, or both, from the cell.

In some embodiments, the cell is a lymphocyte, a dendritic cell, or a macrophage.

In some embodiments, the cell is an activated inflammatory cell.

In some embodiments, the compound is:

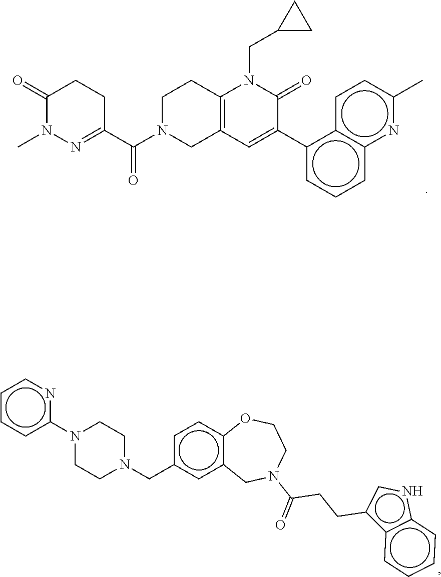

or a combination thereof.

In some embodiments, the fibrotic disease comprises liver fibrosis, skin fibrosis, lung fibrosis, kidney fibrosis, heart fibrosis, or any combination thereof.

In some embodiments, the fibrotic disease is liver fibrosis.

In some embodiments, treating comprises: reducing production of Collagen 1, and of arginase-1, increasing production of IFN-β, or any combination thereof, in the liver of the subject.

In some embodiments, at least one STING inhibitor is:

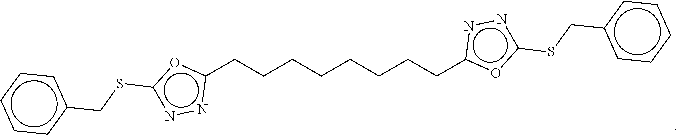

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIGS. 1A-1B include bar graphs showing that Cluster 1 and 9 inhibit tumor necrosis factor alpha (TNFα) while only cluster 1 promotes IL-10 secretion from resting splenocytes activated by the selective STING agonist DMXAA. Splenocytes were recovered from C57BL/6 WT or STING^{-/-} female mice and were incubated (1×10⁶ cells in 0.5 mL of culture media) overnight with a premade mixture of the individual compounds in each cluster (5-500 nM) or vehicle, and DMXAA (10 μM). Next, culture supernatants were collected and TNFα(1A) or IL-10 (1B) levels were determined by standard enzyme-linked immunosorbent assay ELISA (Biolegend).

FIGS. 2A-2D include graphs showing that compounds 5.9 and 7.9 inhibit TNFα and IL-10 secretion from DMXAA-activated splenocytes. Splenocytes were recovered from C57BL/6 WT female mice and incubated (1×10⁶ cells in 0.5 mL of culture media) overnight with the individual compounds from cluster 9 (50-5,000 nM) or vehicle and DMXAA (5-10 μM; 2A-2B) or at 5,000 nM without DMXAA (2C-2D). Next, culture supernatants were collected and TNFα (2A, and 2C) or IL-10 (2B, and 2D) levels were determined by standard ELISA. In 2A-2B, black horizontal line indicates secretion level from vehicle-treated cells.

FIGS. 3A-3H include graphs and an image showing that compounds 2.1, 4.1, and 6.1 inhibit TNFα but promote IL-10 and IFN-beta secretion from DMXAA-activated splenocytes. Splenocytes were recovered from C57BL/6 WT female mice and incubated (1×10⁶ cells in 0.5 mL of culture media) overnight with the individual compounds from cluster 1 (50-5,000 nM) or vehicle and DMXAA (10 μM; 3A, 3C, and 3E) or at 5,000 nM without DMXAA (3B, 3D, and 3F). Next, culture supernatants were collected and TNFα (3A-3B), IL-10 (3C-3D), or IFN-beta (3E-3F) levels were determined by standard ELISA. In 3A, 3C, and 3E, black horizontal line indicates secretion level from vehicle-treated cells. (3G-3H) Biased STING agonists (BiSTs) enhance DMXAA-induced STING signaling. Splenocytes were treated with 25 μg/ml DMXAA and 50 nM of compound 2.1 for 1-4 hr (3G). Then, the cells were analyzed by immunoblotting for phosphorylated STING, TBK-1, IRF3, and NF-kB, or their unphosphorylated controls, or actin as a loading control. Densitometry analysis for phosphorylated STING (p-STING) and STING proteins (3H) is also shown. Results are representative (3G) or mean±SEM (3H) from 3 repeats. One-way ANOVA, and Tukey HSD test *P<0.05.

FIGS. 4A-4C include vertical bar graphs showing that compounds 5.9 and 7.9 inhibit TNFα but promote IL-10 and IFN-beta secretion from lipopolysaccharides (LPS)-activated splenocytes. Splenocytes were recovered from C57BL/6 WT female mice and incubated (1×10⁶ cells in 0.5 mL of culture media) overnight with the individual compounds from cluster 9 (5,000 nM) or vehicle and LPS (1 μg/ml). Next, culture supernatants were collected and TNFα (4A), IL-10 (4B), or IFN-beta (4C) levels were determined by standard ELISA.

FIGS. 5A-5D include bar graphs showing that compound 7.9 inhibits TNFα and IL-10 secretion from DMXAA-activated resolution phase macrophages, while compound 4.1 is a selective inducer of IL-10. Mice were injected with zymosan A (1 mg/mouse), and peritoneal cells thereof were recovered after 66 hrs. Exudate macrophages were isolated and incubated (1×10⁶ cells in 0.5 mL of culture media) overnight with a premade mixture of the individual compounds in clusters 1 and 9 (5,000 nM; 5A and 5C), or the individual compounds (500-5,000 nM), or vehicle and DMXAA (5-10 μM). Next, culture supernatants were collected and TNFα (5A-5B) or IL-10 (5C-5D) levels were determined by standard ELISA (Biolegend).

FIGS. 6A-6D include graphs and an image showing that compound 2.1 activates in vivo the STING-IFN-β axis and enhances biased cytokine production by macrophages. Mice were injected with zymosan A for 66 h. Twenty-four 24 post peritonitis initiation (PPI) the mice were injected with DMSO (0.4%), 4 mg/kg DMXAA or 50 μM compound 2.1 (100 μl/mouse). Macrophages were collected from the spleen (6A-6C), or peritoneum (6D) and incubated for 24 h with LPS (1 μg/ml) or vehicle (6A-6C) or lysed and analyzed by Western blotting immediately for the STING pathway (6D). Culture supernatants from incubated macrophages were analyzed by ELISA for the secretion of TNFα (6A), IL-10 (6B), and IL-6 (6C). Results are mean±SEM of 3 experiments (6A-6C) or representative blots. One-way ANOVA, and Tukey HSD test * P≤0.05, ** P≤0.01.

FIGS. 7A-7E include an image and graphs showing that compound 2.1 facilitates anti-fibrotic responses during the resolution of liver fibrosis. Mice were injected I.P. with the fibrosis-inducing agent CCl₄ twice a week. After 28 days the mice were injected with vehicle (DMSO 0.4%), or 50 μM compound 2.1 (100 ml/mouse). After 2 additional days, livers were collected and analyzed by western blot (7A) for their expression of collagen 1 and the M2 marker arginase 1, as well as β-actin (loading control). Alternatively, livers were analyzed for their mRNA expression of the IFN-responsive genes Ifitml (7B) and Ifitm3 (7C), or the anti-fibrotic protein Rgs2 (7D). Liver interstitial fluids were evaluated for the anti-fibrotic cytokine IL-10 (7E) levels. Results are representative from 4 (A) or mean±SEM of 8 mice (7B-7E). One-way ANOVA, and Tukey HSD test ** P≤0.01, **** P≤0.0001.

FIGS. 8A-8G include graphs and an image showing that compound 2.1 activates cytokine expression and STING signaling in human macrophages in a similar fashion to cGAMP. Human monocytes of the U937 cell line were treated with phorbol 12-myristate 13-acetate (PMA; 50 ng/ml) for 48 hours. Then, differentiated macrophages (4×10⁶ cells) were treated with c-di-GMP (10 μg/ml), c-GAMP (1 μg/ml), compound 2.1 (50 nM) or vehicle for 8 (8A-8F) or 2 (8G) hours. Cells (8A-8D, and 8G) or culture supernatants (8E-8F), were collected and analyzed by RT-PCR, ELISA, and immunoblotting for: TNFα (8A), IL-6 (8B), IL-10 (8C), IFN-β (8D), IL-10 (8E), and TNFα (8F), or STING-IFN-β-RGS2 pathway proteins and actin as a loading control (8G). Results are representative mean±SEM from 3 experiments. One-way ANOVA, and Tukey HSD test ** P≤0.01, P<0.001, **** P≤0.0001.

FIGS. 9A-9E include an image of Western blot analysis and vertical bar graphs showing that compound 2.1 reduces production of fibrosis-related factors. (9A) Western blot analysis showing that treatment with compound 2.1 (c2.1) in a liver fibrosis model results in: reduced production of Collagen 1 (Col-1), and of Arginase 1, and increased production of IFN-β. (9B-9E) Vertical bar graphs showing densitometric analysis of (9A) for Col-1 (9B), Arginase-1 (9C), IFN-β (9D), and alpha smooth muscle actin (αSMA; 9E). (9B-9E) results are mean±SEM. One-way ANOVA, and Tukey HSD test * P≤0.05, ** P≤0.01.

### DETAILED DESCRIPTION

In one aspect of the invention, there is provided a compound, a composition of matter, or a pharmaceutical composition comprising one or more of the following compound(s):

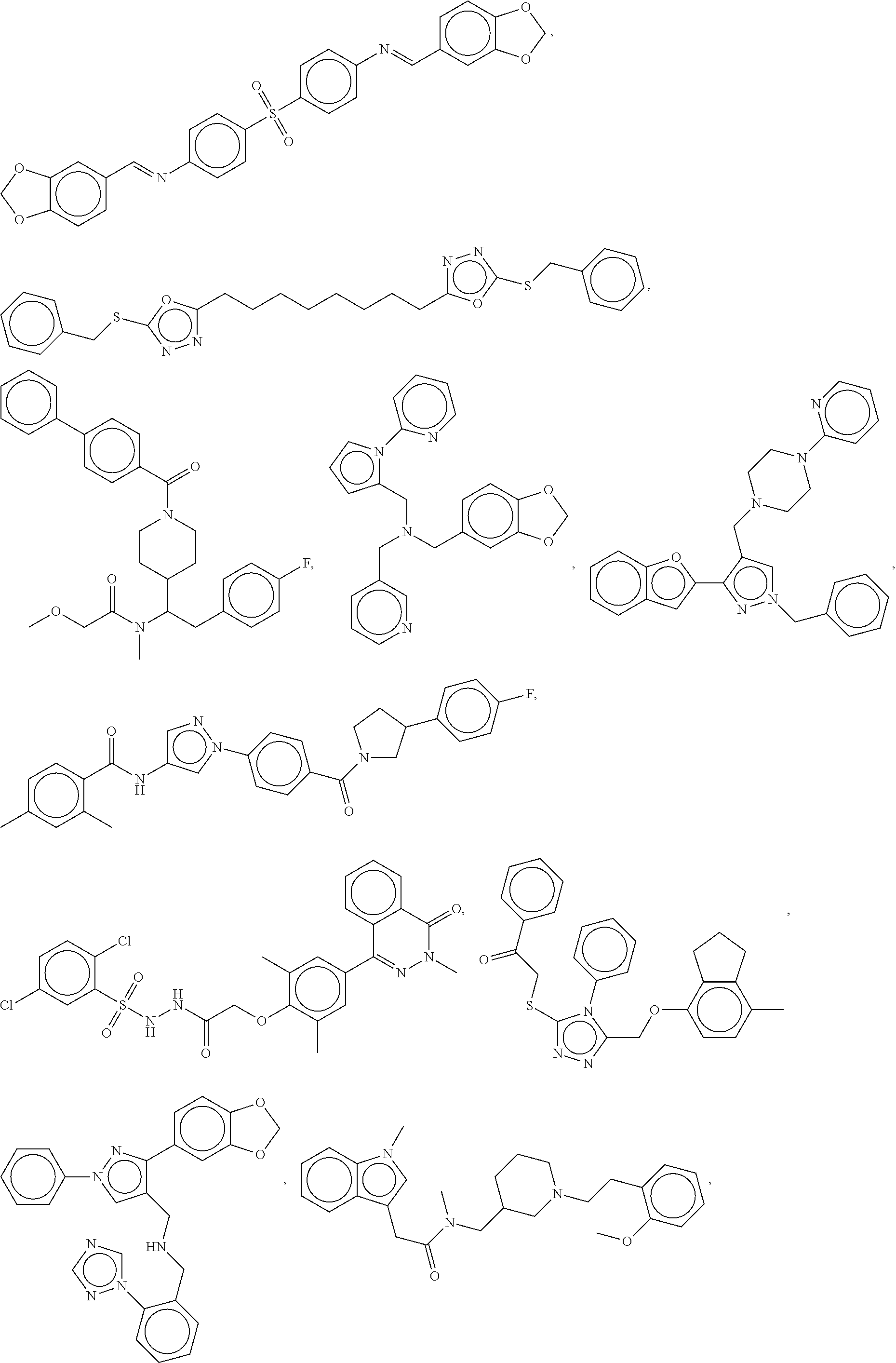

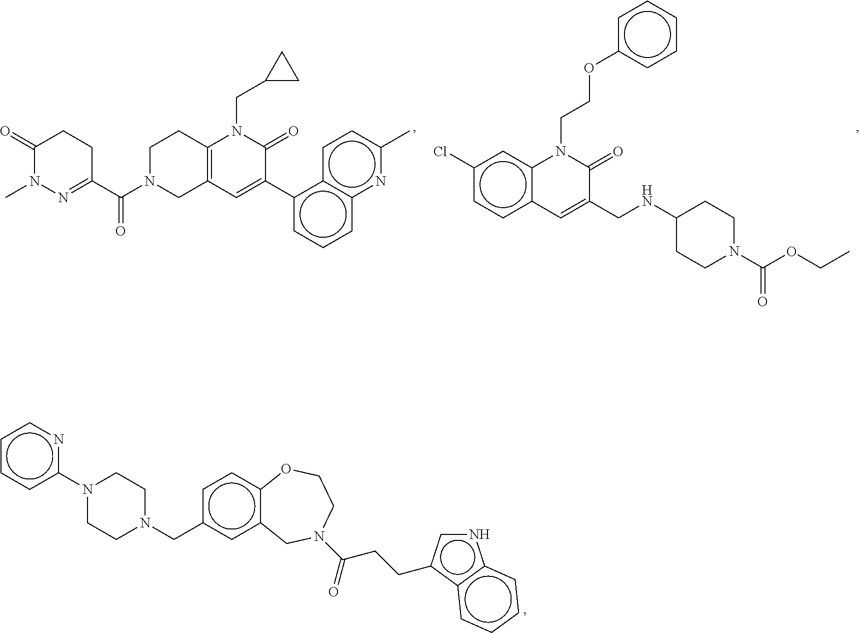

or any combination thereof.

In one embodiment, a compound, a composition of matter, or a pharmaceutical composition is effective in inhibiting STING. In one embodiment. STING is cyclic guanosine monophosphate-adenosine monophosphate synthase (cGAS)-stimulator of interferon genes (STING). In one embodiment, STING signaling pathway is the primary immune response pathway in the cytoplasm.

In one embodiment, a compound, a composition of matter, or a pharmaceutical composition is effective in the treatment of inflammatory diseases and autoimmune diseases. In one embodiment, a compound, a composition of matter, or a pharmaceutical composition is effective in treating neurodegenerative diseases. In one embodiment, a compound, a composition of matter, or a pharmaceutical composition is effective in treating autoimmune diseases. In one embodiment, a compound, a composition of matter, or a pharmaceutical composition is effective in treating neurodegenerative diseases' STING-driven inflammation.

In one embodiment, a compound, a composition of matter, or a pharmaceutical composition is effective in inhibiting the production of type I interferon, decreasing TBK1 phosphorylation, inhibiting palmitoylation or dimerization of human STING, or any combination thereof. In one embodiment, a compound, a composition of matter, or a pharmaceutical composition as described herein competes with the high molecular weight (674 Da), high-affinity endogenous ligand (Kd=4.6 nM) cGAMP.

In one embodiment, a compound, a composition of matter, or a pharmaceutical composition is effective in inhibiting TNF-alpha secretion from a cell. In one embodiment, a compound, a composition of matter, or a pharmaceutical composition is effective in inducing IL-10 secretion. In one embodiment, a compound, a composition of matter, or a pharmaceutical composition is effective in inducing IFN-beta secretion. In one embodiment, a compound, a composition of matter, or a pharmaceutical composition is effective in inducing IFN-beta and IL-10 secretion.

In one embodiment, a compound, a composition of matter, or a pharmaceutical composition is effective in inhibiting IL-10 secretion. In one embodiment, a compound, a composition of matter, or a pharmaceutical composition is effective in inhibiting IFN-beta secretion. In one embodiment, a compound, a composition of matter, or a pharmaceutical composition is effective in inhibiting IFN-beta and IL-10 secretion.

In one embodiment, a compound, a composition of matter, or a pharmaceutical composition is effective for the above indications when contacted with a target cell to be affected. In one embodiment, a compound, a composition of matter, or a pharmaceutical composition is used in a method for treating of inflammatory diseases and autoimmune diseases. In one embodiment, a compound, a composition of matter, or a pharmaceutical composition is used in a method for treating neurodegenerative diseases. In one embodiment, a compound, a composition of matter, or a pharmaceutical composition is used in a method for treating autoimmune diseases. In one embodiment, a compound, a composition of matter, or a pharmaceutical composition is used in a method for treating neurodegenerative diseases' STING-driven inflammation.

In one embodiment, a compound, a composition of matter, or a pharmaceutical composition is used in a method for inhibiting the production of type I interferon, decrease TBK1 phosphorylation, inhibit palmitoylation or dimerization of human STING, or any combination thereof.

According to another aspect, a compound, a composition of matter, or a pharmaceutical composition is used in a method for treating a fibrotic disease in a subject in need thereof.

As used herein, “fibrosis” refers to the formation of excess fibrous connective tissue as a result of the excess deposition of extracellular matrix components, for example collagen. Fibrous connective tissue is characterized by having extracellular matrix (ECM) with a high collagen content. The collagen may be provided in strands or fibers, which may be arranged irregularly or aligned. The ECM of fibrous connective tissue may also include glycosaminoglycans.

As used herein, “excess fibrous connective tissue” refers to an amount of connective tissue at a given location (e.g., a given tissue or organ, or part of a given tissue or organ) that is greater than the amount of connective tissue present at that location in the absence of fibrosis, e.g., under normal, non-pathological conditions. As used herein, “excess deposition of extracellular matrix components” refers to a level of deposition of one or more extracellular matrix components which is greater than the level of deposition in the absence of fibrosis, e.g. under normal, non-pathological conditions.

The cellular and molecular mechanisms of fibrosis are described in Wynn, J. Pathol. (2008) 214 (2): 199-210, and Wynn and Ramalingam, Nature Medicine (2012) 18:1028-1040, which are hereby incorporated by reference in their entirety.

In some embodiments, treating comprises: reducing the production of or enhancing the elimination of Collagen-1 and/or of argniase-1, increasing production of IFN-β, or any combination thereof, in the liver, skin, lung, kidney, heart, or any combination thereof, of the subject.

In some embodiments, treating comprises: reducing the production of or enhancing the elimination of Collagen-1 and/or of argniase-1, increasing production of IFN-β, or any combination thereof, in the liver of the subject.

In some embodiments, production comprises expression. In some embodiments, expression comprises gene and/or mRNA expression level, protein level, or both.

In some embodiments, the method comprises reducing transcription, translation, or both of Collagen-1, arginase-1, or both.

In some embodiments, the method comprises increasing transcription, translation, or both of IFN-β.

Methods and means for determining expression are common and would be apparent to one of ordinary skill in the art of molecular biology and biochemistry. Non-limiting examples of methods of expression determination include but are not limited to, quantitative PCR, NGS, western blot, densitometry, and others.

In some embodiments, fibrotic disease comprises liver fibrosis, skin fibrosis, lung fibrosis, kidney fibrosis, heart fibrosis, or any combination thereof.

In some embodiments, a fibrotic disease comprises or is liver fibrosis.

In hepatic fibrosis, excessive connective tissue accumulates in the liver; this tissue represents scarring in response to chronic, repeated liver cell injury. Commonly, fibrosis progresses, disrupting hepatic architecture and eventually function, as regenerating hepatocytes attempt to replace and repair damaged tissue. When such disruption is widespread, cirrhosis is diagnosed.

Hepatic fibrosis is suspected if patients have known chronic liver disease (e.g., chronic viral hepatitis C [HCV] or chronic hepatitis B [HBV], alcoholic liver disease) or if results of liver blood tests are abnormal; in such cases, tests are done to check for fibrosis and, if fibrosis is present, to determine its severity (stage). Knowing the stage of fibrosis can guide medical decisions. For example, screening for hepatocellular carcinoma and for gastroesophageal varices is indicated if cirrhosis is confirmed, but it is generally not indicated for mild or moderate fibrosis. Assessment of the degree of hepatic fibrosis helps assess the prognosis of patients with chronic viral hepatitis. However, since the widespread availability of direct-acting antiviral drugs, knowing the degree of fibrosis has become much less important in deciding when to initiate antiviral therapy.

Tests used to stage fibrosis include conventional imaging tests, blood tests, liver biopsy, and newer noninvasive imaging tests that assess liver stiffness.

Conventional imaging tests: Conventional imaging tests include ultrasonography, CT, and MRI. These tests can detect evidence of cirrhosis and portal hypertension, such as liver surface nodularity, splenomegaly, and varices. However, they are not sensitive for moderate or even advanced fibrosis and may fail to detect some cases of cirrhosis if splenomegaly and varices are absent. Although fibrosis may appear as altered echogenicity on ultrasonography or heterogeneity of signal on CT, these findings are nonspecific and may indicate only liver parenchymal fat.

Noninvasive imaging assessment of fibrosis: Newer acoustic technologies can increase the accuracy of ultrasonography, CT, and MRI for detecting fibrosis or early cirrhosis; they include: transient elastography, acoustic radiation force impulse imaging, two-dimensional shear wave elastography, and magnetic resonance elastography.

For these tests, acoustic vibrations are applied to the abdomen with a probe. How rapidly these vibrations are transmitted through liver tissue indicates how stiff (ie, fibrosed) the liver is. However, certain other conditions besides fibrosis also increase liver stiffness, including severe active hepatitis, increased right heart pressures, and the postprandial state. Also, these techniques have not been validated well in pregnancy, sustained virologic response after HCV treatment, and rare liver disorders. Thus, the use of these techniques is typically not recommended in patients with one of these conditions.

Liver biopsy remains the gold standard for diagnosing and staging hepatic fibrosis and for diagnosing the underlying liver disorder causing fibrosis. However, liver biopsy is invasive, resulting in a 10 to 20% risk of minor complications (e.g., postprocedural pain) and a 0.5 to 1% risk of serious complications (e.g., significant bleeding). Also, liver biopsy is limited by sampling error and imperfect interobserver agreement in interpretation of histologic findings. Thus, liver biopsy may not always be done. Liver biopsy is usually not done solely for staging of hepatic fibrosis unless noninvasive tests do not help establish the diagnosis (e.g., because different noninvasive tests yield discordant results) or for clinical trials.

Blood tests are included in clinical models (e.g., APRI index, BARD score, FIB-4 score, nonalcoholic fatty liver disease [NAFLD] fibrosis score), which combine commonly available tests (AST, ALT, platelet count, albumin, INR) with demographic and clinical information (e.g., age, body mass index [BMI], diabetes/impaired fasting glucose). Some commercially available panels (e.g., FibroTest [known as Fibrosure in the US], Hepascore, European Liver Fibrosis panel [ELF]) combine indirect markers (e.g., serum bilirubin) and direct markers of hepatic function. Direct markers are substances involved in the pathogenesis of extracellular matrix deposition or cytokines that induce extracellular matrix deposition. These models and panels are best used to distinguish between 2 levels of fibrosis: absent to minimal vs moderate to severe; they do not accurately differentiate between degrees of moderate to severe fibrosis. Therefore, if fibrosis is suspected, one approach is to start with one of these panels and then do the new noninvasive imaging assessments of fibrosis, reserving liver biopsy as a last resort.

In one embodiment, a compound, a composition of matter, or a pharmaceutical composition is used in a method for inhibiting TNF-alpha secretion from a cell. In one embodiment, a compound, a composition of matter, or a pharmaceutical composition is used in a method for inducing IL-10 secretion in a cell. In one embodiment, a compound, a composition of matter, or a pharmaceutical composition is used in a method for inducing IFN-beta secretion. In one embodiment, a compound, a composition of matter, or a pharmaceutical composition is used in a method for inducing IFN-beta and IL-10 secretion in a cell.

In one embodiment, a compound, a composition of matter, or a pharmaceutical composition is used in a method for inhibiting IL-10 secretion in a cell. In one embodiment, a compound, a composition of matter, or a pharmaceutical composition is used in a method for inhibiting IFN-beta secretion in a cell. In one embodiment, a compound, a composition of matter, or a pharmaceutical composition is used in a method for inhibiting IFN-beta and IL-10 secretion in a cell.

In one embodiment, a cell is the target of a compound, a composition of matter, or a pharmaceutical composition, as described herein. In one embodiment, a tissue comprising a cell as described herein is the target of a compound, a composition of matter, or a pharmaceutical composition, as described herein. In one embodiment, an organ comprising a cell as described herein is the target of a compound, a composition of matter, or a pharmaceutical composition, as described herein.

In one embodiment, the cell is a lymphocyte. In one embodiment, the cell is a dendritic cell. In one embodiment, the cell is a macrophage. In one embodiment, the cell is an activated inflammatory cell.

In one embodiment, the compound/s to be used in the present invention is/are listed in table 1 and table 2. In one embodiment, a composition or a pharmaceutical composition as described herein comprises the compound or any combination of compounds listed in table 1 and table 2.

TABLE 1

| Cluster | Structure | CHEM_NAME |
|---|---|---|
| 1.1 | 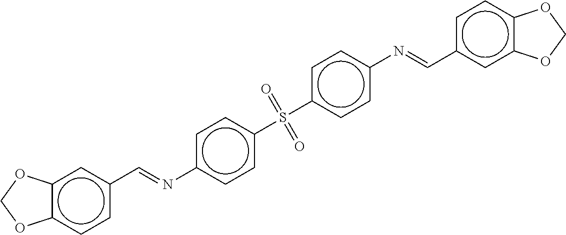 | 4- (PIPERONYLIDENE- AMINO) PHENYL SULFONE |
| 2.1 | 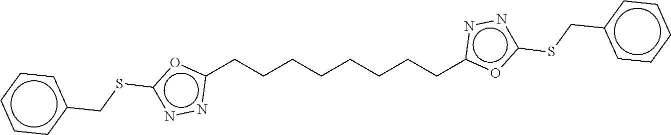 | 2-(benzylsulfanyl)-5-{8- [5-(benzylsulfanyl)- 1,3,4-oxadiazol-2- yl]octyl}-1,3,4- oxadiazole |
| 3.1 | 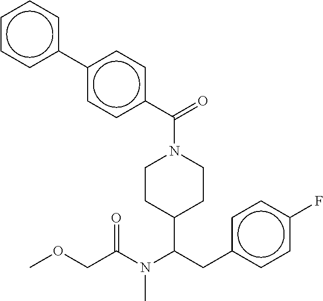 | N-[1-[1-(4- biphenylylcarbonyl)-4- piperidinyl]-2-(4- fluorophenyl)ethyl]-2- methoxy-N- methylacetamide |
| 4.1 | 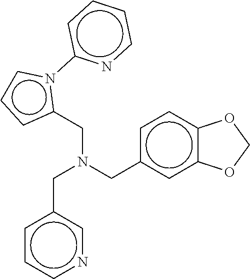 | (1,3-benzodioxol-5- ylmethyl)(pyridin-3- ylmethyl)[(1-pyridin-2- yl-1H-pyrrol-2- yl)methyl]amine |
| 5.1 | 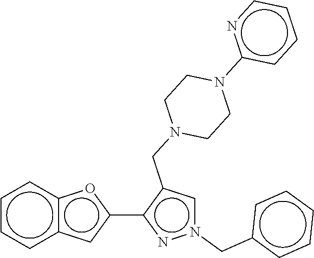 | 1-{[3-(1-benzofuran-2- yl)-1-benzyl-1H-pyrazol- 4-yl]methyl}-4-(2- pyridinyl)piperazine |
| 6.1 | 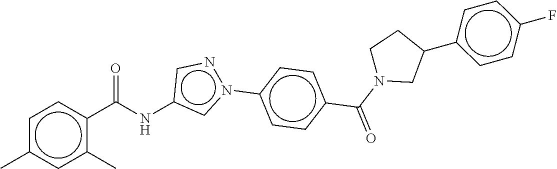 | N-[1-(4-{[3-(4- fluorophenyl)-1- pyrrolidinyl]carbonyl} phenyl)-1H-pyrazol-4-yl]- 2,4-dimethylbenzamide |

TABLE 2

| Cluster | Structure | CHEM_NAME |
|---|---|---|
| 1.9 | 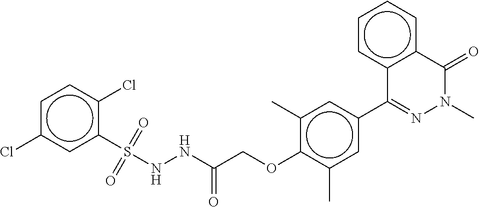 | 2,5-dichloro-N′- {[2,6-dimethyl-4-(3- methyl-4-oxo-3,4- dihydro-1- phthalazinyl)phenoxy] acetyl}benzenesulfono- hydrazide |
| 2.9 | 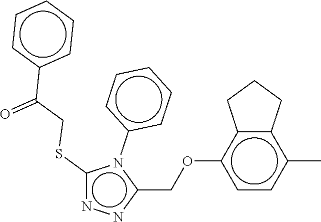 | 2-[(5-{[(7-methyl- 2,3-dihydro-1H- inden-4- yl)oxy]methyl}-4- phenyl-4H-1,2,4- triazol-3-yl)sulfanyl]- 1-phenylethanone |
| 3.9 | 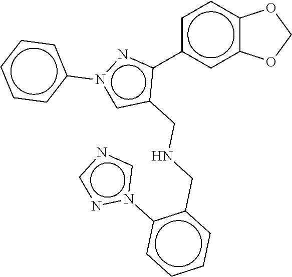 | 1-[3-(1,3- benzodioxol-5-yl)-1- phenyl-1H-pyrazol- 4-yl]-N-[2-(1H-1,2,4- triazol-1- yl)benzyl]methanamine |
| 4.9 | 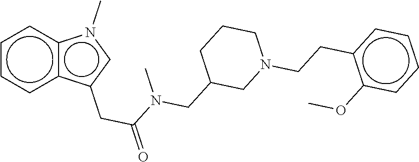 | N-({1-[2-(2- methoxyphenyl)ethyl 1-3- piperidinyl}methyl)- N-methyl-2-(1- methyl-1H-indol-3- yl)acetamide |
| 5.9 | 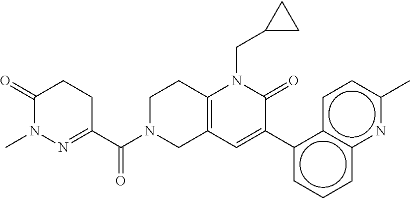 | 1- (cyclopropylmethyl)- 6-[(1-methyl-6-oxo- 1,4,5,6-tetrahydro-3- pyridazinyl)carbonyl]- 3-(2-methyl-5- quinolinyl)-5,6,7,8- tetrahydro-1,6- naphthyridin-2(1H)- one |
| 6.9 | 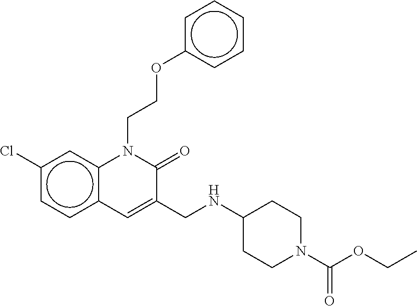 | ethyl 4-({[7-chloro-2- oxo-1-(2- phenoxyethyl)-1,2- dihydro-3- quinolinyl]methyl} amino)-1- piperidinecarboxylate |
| 7.9 | 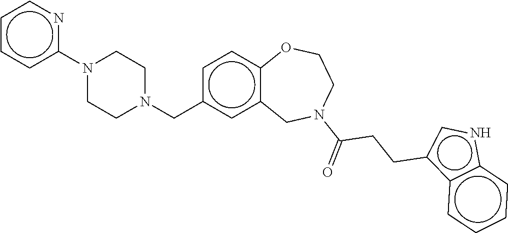 | 4-[3-(1H-indol-3- yl)propanoyl]-7-{[4- (2-pyridinyl)-1- piperazinyl]methyl}- 2,3,4,5-tetrahydro- 1,4-benzoxazepine |

In some embodiments, a STING inhibitor or inhibitors as described herein is/are used for adjuvant therapy to combat retrovirus infection, including SARS-COV2. In some embodiments, a STING inhibitor or inhibitors as described herein is/are used for the treatment of an autoimmune disease. In some embodiments, a STING inhibitor or inhibitors as described herein is/are used for the treatment of a systemic autoimmune disease. In some embodiments, a STING inhibitor or inhibitors as described herein is/are used for the treatment of a systemic chronic autoimmune disease. In some embodiments, a STING inhibitor or inhibitors as described herein is/are used for the treatment of lupus. In some embodiments, a STING inhibitor or inhibitors as described herein is/are used for the treatment of an inflammatory disease.

In some embodiments, the compound of the invention comprises any one of the compounds disclosed herein, including any salt thereof, any tautomer thereof, or a combination thereof. In some embodiments, the salt, the tautomer, or both, of the compound is a pharmaceutically acceptable salt, tautomer, or both.

In some embodiments, there is provided a composition comprising the compound of the invention, and an acceptable carrier.

### Pharmaceutical Composition

In another aspect of the invention disclosed herein, there is a pharmaceutical composition comprising the compound of the invention, a pharmaceutically acceptable salt thereof or both.

Non-limiting examples of pharmaceutically acceptable salts include but are not limited to: acetate, aspartate, benzenesulfonate, benzoate, bicarbonate, carbonate, halide (such as bromide, chloride, iodide, fluoride), bitartrate, citrate, salicylate, stearate, succinate, sulfate, tartrate, decanoate, edetate, fumarate, gluconate, and lactate or any combination thereof.

In some embodiments, the pharmaceutical composition comprises the compound of the invention and a pharmaceutically acceptable carrier. In some embodiments, the pharmaceutical composition comprises a therapeutically effective amount of the compound of the invention and the pharmaceutically acceptable carrier.

For example, the term “pharmaceutically acceptable” can mean approved by a regulatory agency of the Federal or a state government or listed in the U.S. Pharmacopcia or other generally recognized pharmacopcia for use in animals, and more particularly in humans. In some embodiments, the compound of the invention is referred to herein as an active ingredient of a pharmaceutical composition.

In some embodiments, the pharmaceutical composition as described herein is a topical composition. In some embodiments, the pharmaceutical composition is an oral composition. In some embodiments, the pharmaceutical composition is an injectable composition. In some embodiments, the pharmaceutical composition is for systemic use.

In some embodiments, the pharmaceutical composition is any of an emulsion, a liquid solution, a gel, a paste, a suspension, a dispersion, an ointment, a cream, or a foam.

As used herein, the term “carrier” refers to a diluent, adjuvant, excipient, or vehicle with which the active ingredient is administered. Such carriers can be sterile liquids, such as water-based and oils, including those of petroleum, animal, vegetable, or synthetic origin, such as peanut oil, soybean oil, mineral oil, sesame oil and the like, polyethylene glycols, glycerin, propylene glycol or other synthetic solvents.

Other non-limiting examples of carriers include, but are not limited to: terpenes derived from Cannabis, or total terpene extract from Cannabis plants, terpenes from coffee or cocoa, mint-extract, eucalyptus-extract, citrus-extract, tobacco-extract, anis-extract, any vegetable oil, peppermint oil, d-limonene, b-myrcene, a-pinene, linalool, anethole, a-bisabolol, camphor, b-caryophyllene and caryophyllene oxide, 1,8-cineole, citral, citronella, delta-3-carene, farnesol, geraniol, indomethacin, isopulegol, linalool, unalyl acetate, b-myrcene, myrcenol, 1-menthol, menthone, menthol and neomenthol, oridonin, a-pinene, diclofenac, nepafenac, bromfenac, phytol, terpincol, terpinen-4-ol, thymol, and thymoquinone. One skilled in the art will appreciate that a particular carrier used within the pharmaceutical composition of the invention may vary depending on the route of administration.

In some embodiments, the carrier improves the stability of the active ingredient in a living organism. In some embodiments, the carrier improves the stability of the active ingredient within the pharmaceutical composition. In some embodiments, the carrier enhances the bioavailability of the active ingredient.

Water may be used as a carrier such as when the active ingredient has sufficient aqueous solubility, so as to be administered intravenously. Saline solutions and aqueous dextrose and glycerol solutions can also be employed as liquid carriers, particularly for injectable solutions.

In some embodiments, the carrier is a liquid carrier. In some embodiments, the carrier is an aqueous carrier.

Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol, and the like. The composition, if desired, can also contain minor amounts of wetting or emulsifying agents, or pH buffering agents such as acetates, citrates, or phosphates. Antibacterial agents such as benzyl alcohol or methyl parabens; antioxidants such as ascorbic acid or sodium bisulfite; and agents for the adjustment of tonicity, such as sodium chloride or dextrose are also envisioned. The carrier may comprise, in total, from 0.1% to 99.99999% by weight of the composition/s or the pharmaceutical composition/s presented herein.

In some embodiments, the pharmaceutical composition includes incorporation of any one of the active ingredients into or onto particulate preparations of polymeric compounds such as polylactic acid, polyglycolic acid, hydrogels, etc., or onto liposomes, microemulsions, micelles, unilamellar or multilamellar vesicles, erythrocyte ghosts, or spheroplasts. Such compositions may influence the physical state, solubility, stability, rate of in vivo release, and rate of in vivo clearance.

In some embodiments, the pharmaceutical composition is a liquid at a temperature between 15 to 45° C. In some embodiments, the pharmaceutical composition is a solid at a temperature between 15 to 45° C. In some embodiments, the pharmaceutical composition is a semi-liquid at a temperature between 15 to 45° C. It should be understood that the term “semi-liquid”, is intended to mean materials which are flowable under pressure and/or shear force. In some embodiments, semi-liquid compositions include creams, ointments, gel-like materials, and other similar materials. In some embodiments, the pharmaceutical composition is a semi-liquid composition, characterized by viscosity in a range from 31,000-800,000 cps.

Non-limiting examples of carriers for pharmaceutical compositions being in the form of a cream include but are not limited to: non-ionic surfactants (e.g., glyceryl monolinoleate glyceryl monooleate, glyceryl monostearate lanolin alcohols, lecithin mono- and di-glycerides poloxamer polyoxyethylene 50 stearate, and sorbitan trioleate stearic acid), anionic surfactants (e.g. pharmaceutically acceptable salts of fatty acids such as stearic, oleic, palmitic, and lauric acids), cationic surfactants (e.g. pharmaceutically acceptable quaternary ammonium salts such as benzalkonium chloride, benzethonium chloride, and cetylpyridinium chloride) or any combination thereof.

In some embodiments, the pharmaceutical composition being in the form of a cream further comprises a thickener.

Non-limiting examples of thickeners include, but are not limited to microcrystalline cellulose, a starch, a modified starch, gum tragacanth, gelatin, and a polymeric thickener (e.g., polyvinylpyrrolidone) or any combination thereof.

In some embodiments, the pharmaceutical composition comprising the compound of the invention is in a unit dosage form. In some embodiments, the pharmaceutical composition is prepared by any of the methods well-known in the art of pharmacy. In some embodiments, the unit dosage form is in the form of a tablet, capsule, lozenge, wafer, patch, ampoule, vial, or pre-filled syringe.

In addition, in vitro assays may optionally be employed to help identify optimal dosage ranges. The precise dose to be employed in the formulation will also depend on the route of administration, and the nature of the disease or disorder, and should be decided according to the judgment of the practitioner and each patient's circumstances. Effective doses can be extrapolated from dose-response curves derived from in-vitro or in-vivo animal model test bioassays or systems. In some embodiments, the effective dose is determined as described hereinabove.

In another embodiment, the pharmaceutical composition of the invention is administered in any conventional oral, parenteral, or transdermal dosage form.

As used herein, the terms “administering”, “administration”, and like terms refer to any method which, in sound medical practice, delivers a composition containing an active agent to a subject in such a manner as to provide a therapeutic effect.

In some embodiments, the pharmaceutical composition is administered via oral (i.e., enteral), rectal, vaginal, topical, sublingual, buccal, nasal, ophthalmic, transdermal, subcutaneous, intramuscular, intraperitoneal, intrathecal, or intravenous routes of administration. The route of administration of the pharmaceutical composition will depend on the disease or condition to be treated. Suitable routes of administration include but are not limited to, parenteral injections, e.g., intradermal, intravenous, intramuscular, intralesional, subcutaneous, intrathecal, and any other mode of injection as known in the art. In addition, it may be desirable to introduce the pharmaceutical composition of the invention by any suitable route, including intraventricular and intrathecal injection; intraventricular injection may be facilitated by an intraventricular catheter, for example, attached to a reservoir. Pulmonary administration can also be employed, e.g., by use of an inhaler or nebulizer.

In some embodiments, the pharmaceutical composition is in a form of, for example, and not by way of limitation, an ointment, cream, gel, paste, foam, aerosol, suppository, pad, or gelled stick.

In some embodiments, for oral applications, the pharmaceutical composition is in the form of a tablet or a capsule, which can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate; or a glidant such as colloidal silicon dioxide. When the dosage unit form is a capsule, it can contain, in addition to materials of the above type, a liquid carrier such as fatty oil. In addition, dosage unit forms can contain various other materials which modify the physical form of the dosage unit, for example, coatings of sugar, shellac, or other enteric agents. In some embodiments, the tablet of the invention is further film-coated. In some embodiments, oral application of the pharmaceutical composition or of the kit is in a form of a drinkable liquid. In some embodiments, oral application of the pharmaceutical composition or of the kit is in a form of an edible product.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous, and intraperitoneal injection purposes.

In some embodiments, a method as described herein comprises administering the pharmaceutical composition of the invention at least 1 time, at least 2 times, at least 3 times, at least 4 times, at least 5 times, at least 7 times, or at least 10 times per day or per week or per month, or any value and range therebetween. Each possibility represents a separate embodiment of the invention. In some embodiments, the method comprises administering the composition or the combination of the invention 1-2 times per day or per week or per month, 1-3 times per day or per week or per month, 1-4 times per day or per week or per month, 1-5 times per day, 1-7 times per day or per week or per month, 2-3 times per day or per week or per month, 2-4 times per day or per week or per month, 2-5 times per day or per week or per month, 3-4 times per day or per week or per month, 3-5 times per day or per week or per month, or 5-7 times per day or per week or per month. Each possibility represents a separate embodiment of the invention.

In some embodiments, the method comprises administering the pharmaceutical composition of the invention to the subject at a daily or weekly or monthly dosage of 0.05 to 20 mg/kg, 0.05 to 0.1 mg/kg, 0.1 to 0.3 mg/kg, 0.3 to 0.5 mg/kg, 0.5 to 0.8 mg/kg, 0.8 to 1 mg/kg, 1 to 2 mg/kg, 2 to 5 mg/kg, 5 to 10 mg/kg, 10 to 15 mg/kg, 15 to 20 mg/kg including any range or value therebetween.

It should be apparent to one skilled in the art, that for example in-vitro and in-vivo assays may optionally be employed to help identify optimal dosage ranges. The precise dose to be employed in the formulation will also depend on the route of administration, and the nature of the disease or disorder, and should be decided according to the judgment of the practitioner and each patient's circumstances. Effective doses can be extrapolated from dose-response curves derived from in-vitro or in-vivo animal model test bioassays or systems.

In some embodiments, the subject is a mammal. In some embodiments, the subject is a lab animal. In some embodiments, the subject is a pet. In some embodiments, the subject is a rodent. In some embodiments, the subject is a farm animal. In some embodiments, the subject is a human subject.

In some embodiments, the composition of the present invention is administered in a therapeutically safe and effective amount. As used herein, the term “safe and effective amount” refers to the quantity of a component that is sufficient to yield a desired therapeutic response without undue adverse side effects, including but not limited to toxicity, such as calcemic toxicity, irritation, or allergic response, commensurate with a reasonable benefit/risk ratio when used in the presently described manner. The actual amount administered, and the rate and time course of administration will depend on the nature and severity of the condition being treated. Prescription of treatment, e.g., decisions on dosage, timing, etc., is within the responsibility of general practitioners or specialists, and typically takes account of the disorder to be treated, the condition of the individual patient, the site of delivery, the method of administration and other factors known to practitioners. Examples of techniques and protocols can be found in Remington: The Science and Practice of Pharmacy, 21st Ed., Lippincott Williams & Wilkins, Philadelphia, Pa., (2005).

In some embodiments, the effective amount or dose of the active ingredient can be estimated initially from in vitro assays. In one embodiment, a dose can be formulated in animal models and such information can be used to determine useful doses more accurately in humans.

In one embodiment, toxicity and therapeutic efficacy of the active ingredients described herein can be determined by standard pharmaceutical procedures in vitro, in cell cultures, or in experimental animals. In one embodiment, the data obtained from these in vitro and cell culture assays and animal studies can be used in formulating a range of dosages for use in humans. In one embodiment, the dosages may vary depending on the dosage form employed and the route of administration utilized. In one embodiment, the exact formulation, route of administration, and dosage can be chosen by the individual physician in view of the patient's condition. [See e.g., Goodman and Gilman's The Pharmacological Basis of Therapeutics, 13th Ed., McGraw-Hill/Education, New York, NY (2017)].

In some embodiments, the method for treating as described herein includes reducing the severity and/or reducing a side effect associated with the disease.

In some embodiments, the compound of the invention has IC50 in inhibiting STING activity between 0.1 and 50 μM, between 1 and 5 nM, between 5 and 10 nM, between 10 and 50 nM, between 50 and 100 nM, between 100 and 500 nM, between 500 nM and I μM, between 1 and 5 μM, between 5 and 10 μM, including any value therebetween.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

### General

It is noted that as used herein and in the appended claims, the singular forms “a,” “an,” and “the” include plural referents unless the context clearly dictates otherwise. Thus, for example, reference to “a polynucleotide” includes a plurality of such polynucleotides, and reference to “the polypeptide” includes reference to one or more polypeptides and equivalents thereof known to those skilled in the art, and so forth. It is further noted that the claims may be drafted to exclude any optional element. As such, this statement is intended to serve as an antecedent basis for use of such exclusive terminology as “solely,” “only” and the like in connection with the recitation of claim elements or use of a “negative” limitation.

In those instances where a convention analogous to “at least one of A, B, and C, etc.” is used, in general, such construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological, and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization-A Laboratory Course Manual” CSHL Press (1996); all of which are incorporated by reference. Other general references are provided throughout this document.

### Materials and Methods

### In Silico Design

To minimize the screen for STING targeting molecules, the compounds, and their respective structures were provided by the compound libraries from the Grand Israel National Center for Personalized Medicine (G-INCPM) at the Weizmann Institute of Science. The structures were screened for their ability to reduce the free energy of the STING dimer by binding to its connector helix and loop. The top-scored 50 compounds were clustered into 10 clusters by the G-INCPM based on structure similarity. The clusters were pooled into 7 batches that were supplied by G-INCPM. The clusters were examined for their regulation of TNFα and IL-10 secretion by unchallenged splenocytes or inflammatory macrophages.

### Cell Isolation and Treatment

Splenocytes were isolated from the spleens of unchallenged C57BL/6 WT or STING-KO mice by meshing against a grid. Peritoneal macrophages were isolated from mice undergoing peritonitis for 66 hr by washing with 5 ml PBS. The cellular exudate was stained with phycoerythrin (PE)-conjugated anti-F4/80 antibodies (bioLegend, CA, USA) and macrophages were isolated using magnetic beads (STEMCELL Technologies Inc, Vancouver, Canada). Isolated splenocytes or macrophages (0.5-1×10⁶ cells per well) were incubated with 50-5,000 nM of individual substances or pooled clusters thereof. Then, the cells were treated with vehicle or 5-10 μM DMXAA (Cayman Chemical, MI, USA) to activate STING. After 24 hr, culture supernatants were collected and evaluated for levels of: TNFα, IFN-β and/or IL-10 using, standard ELISA (BioLegend, CA, USA).

### Mouse Liver Fibrosis Model

Male C57BL/6 mice were randomly assigned to experimental groups at approximately eight weeks old. Mice were injected I.P. with CCl₄ (1 ml/kg) three times a week for six weeks with 0.2 ml of a 10 percent CCl₄ in corn oil. Forty-eight hours before sacrifice, the vehicle group was injected I.P. with CCL4, while the treatment group was injected with CCl₄ and 50 μM of compound 2.1. Then the liver was collected in a RIPA buffer for Western blot analysis.

### Western Blot Analysis

Protein extracts of liver tissue were separated by sodium dodecyl sulfate polyacrylamide gel electrophoresis (SDS-PAGE), transferred to PVDF membranes (Bio-Rad Laboratories, Inc.), blocked with 5% skimmed milk powder, and immunoblotted with antibodies targeting: IFN-β, arginase 1, collagen 1, or β-actin. Then, the membranes were washed, incubated with the appropriate horseradish peroxidase (HRP)-conjugated secondary antibody, and developed using WesternBright™ ECL (Advansta Inc). Band density was quantified with FIJI ImageJ software.

### Example 1

### Cluster 1 and 9 Show STING Modulation Capacity

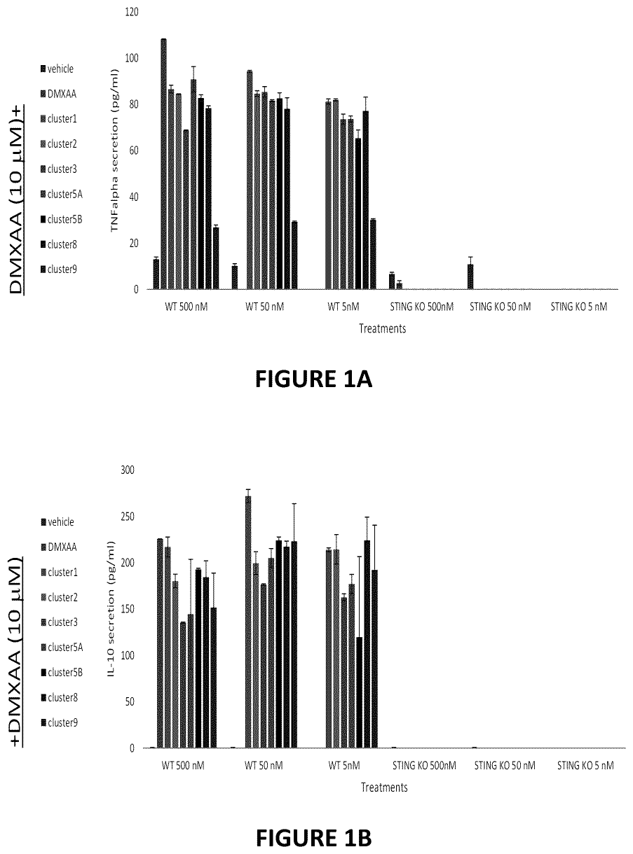

To test which cluster(s) included compounds that modulate STING-mediated cytokine production, wild-type (WT) or STING knockout (−/−) splenocytes were incubated with the selective murine STING agonist DMXAA alone or with 5-500 nM of the pooled individual compounds in each cluster as indicated. The results (FIG. 1) show inhibition of DMXAA-induced TNFα production by all clusters, with cluster 9 exhibiting the highest inhibition and at the lowest concentrations (5-500 nM). Notably, only cluster 1 showed IL-10 stimulating activity (at 50 nM) while cluster 9 inhibited IL-10 induction by DMXAA.

### Example 2

### Compounds 5.9, 4.9, 6.1, and 2.1 are the Best STING Inhibitors at 5,000 nM

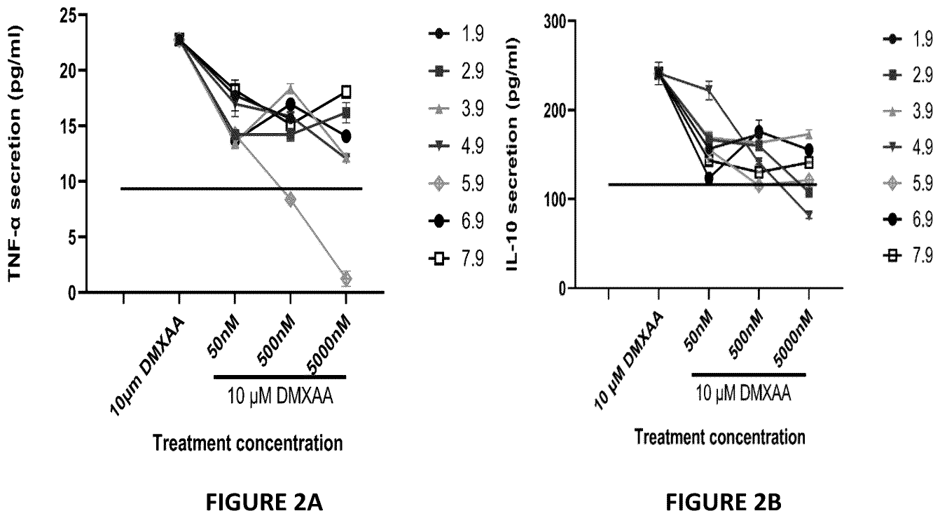

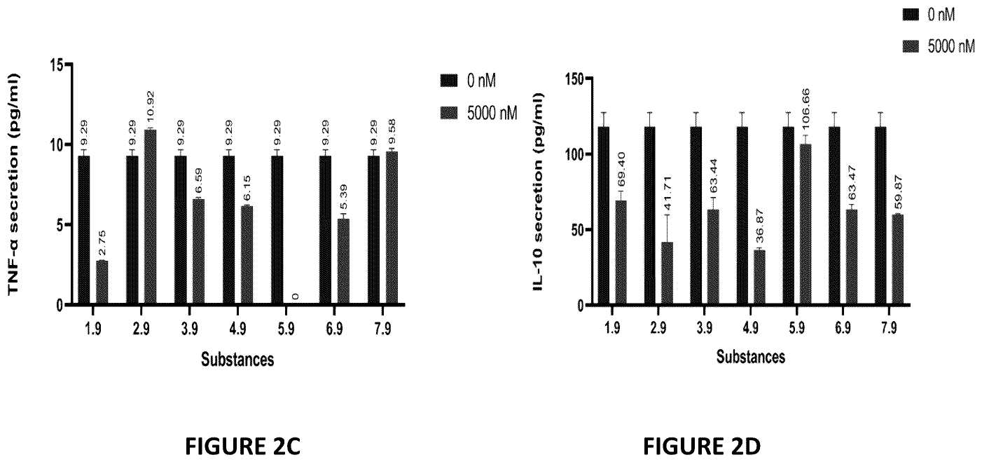

Next, the individual compounds in clusters 1 and 9 were tested at 50-5,000 nM. The first experiment was performed in resting splenocytes as detailed above. The results (FIG. 2) showed several compounds from clusters 1 and 9 inhibited DMXAA-induced TNFα with compounds 5.9, 4.9, 6.1, and 2.1 doing so at lower concentrations. Compound 5.9 was the best inhibitor of TNFα secretion and inhibited it also in the absence of DMXAA stimulation.

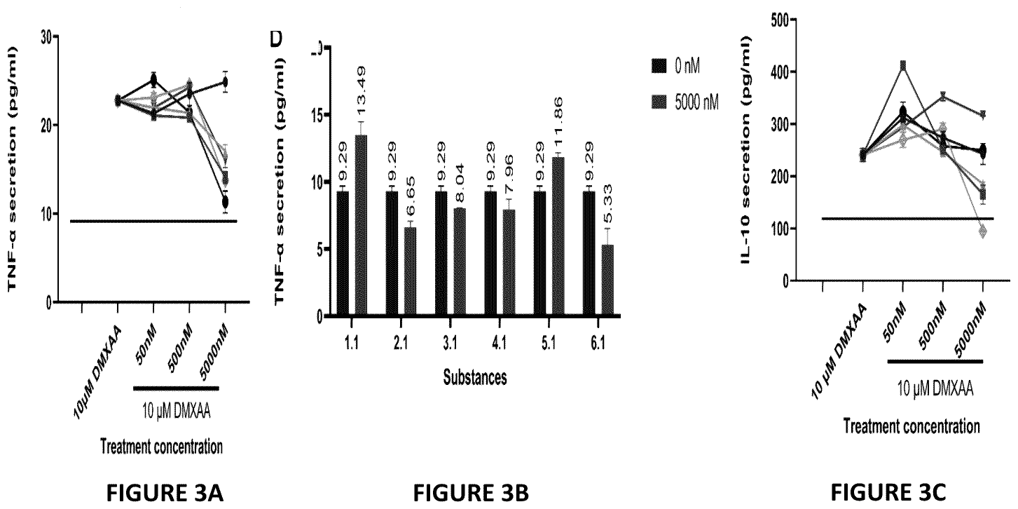

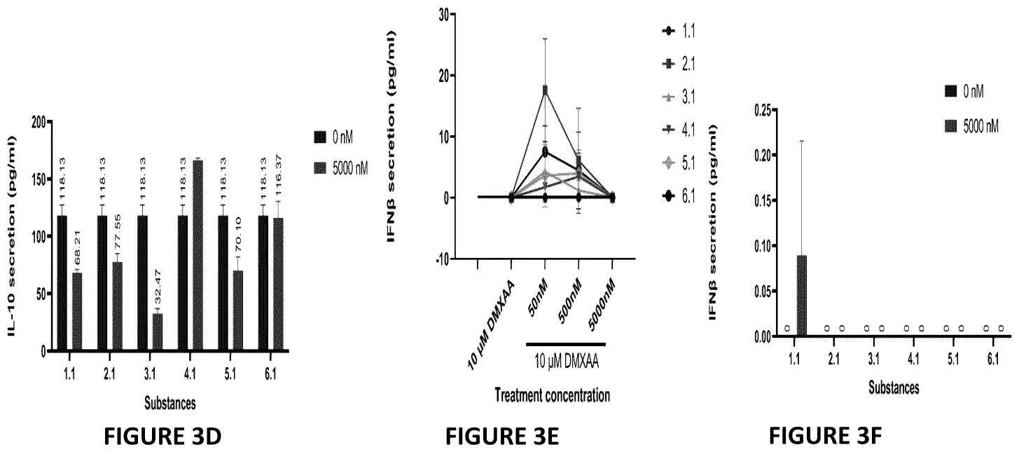

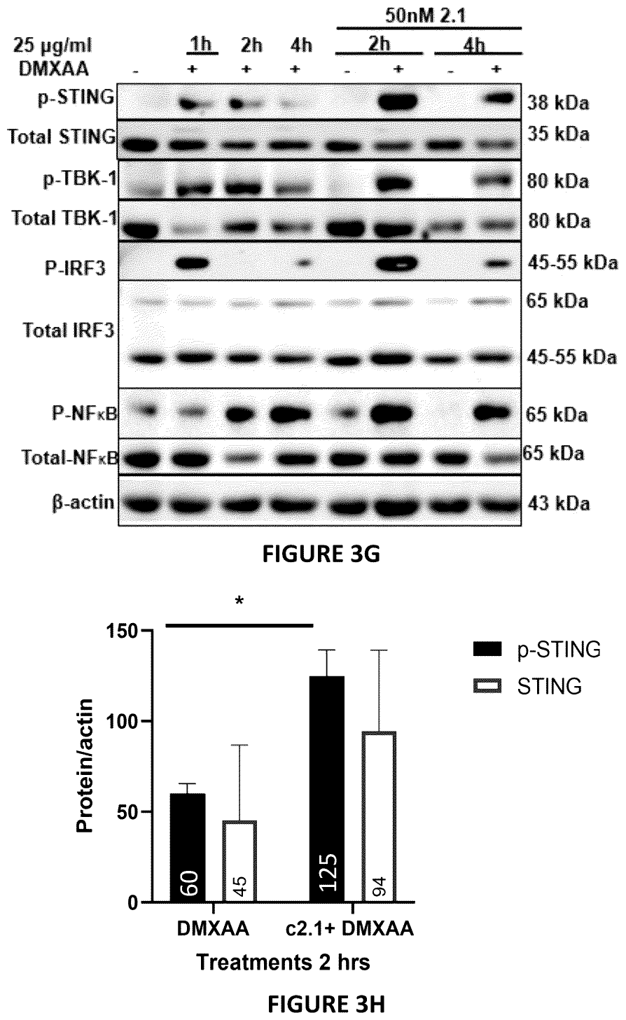

When examining the effect of the compounds on IL-10 and IFN-β secretion, it was found that only the compounds from cluster 1 induced IL-10 and IFN-β, and most of them showed a reversed dose-response that resulted in inhibition of IL-10 production at 5,000 nM. The most potent inducer of IL-10 and IFN-β was compound 2.1, with 6.1 to follow (FIG. 3). Notably, compound 4.1 induced IL-10 at all concentrations, and was the only compound that induced IL-10 (at 5,000 nM) when applied without DMXAA.

### Example 3

### Compound 5.9 Enhances IL-10 and IFN-β, but not TNFα Production by LPS-Stimulated Splenocytes

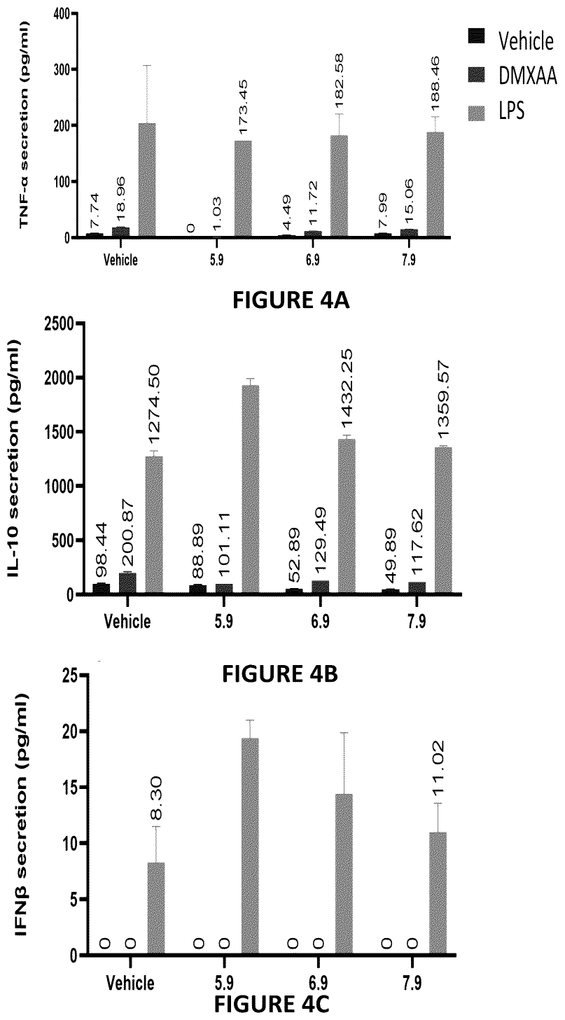

STING was previously shown to be essential for LPS-induced IRF3 activation and IFN-β production. Therefore, further examination of whether the putative STING antagonists affect LPS-induced cytokine production was carried out. It was found that LPS induced higher levels of all cytokines than DMXAA and that compound 5.9 induced a significant upregulation of IFN-β and IL-10, while reducing TNFα (FIG. 4). Compounds 6.9 and 7.9 had much milder effects on LPS-induced cytokine secretion.

### Example 4

### Compound 7.9 is a Very Efficient STING Inhibitor in Resolution Phase Macrophages, Whereas Compound 4.1 is a Biased Antagonist for TNFα

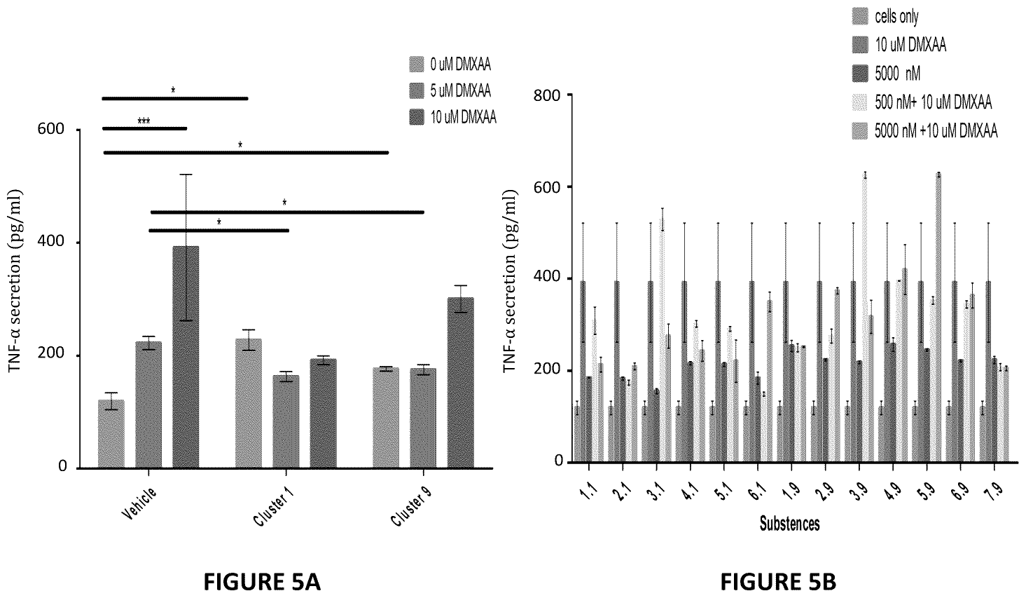

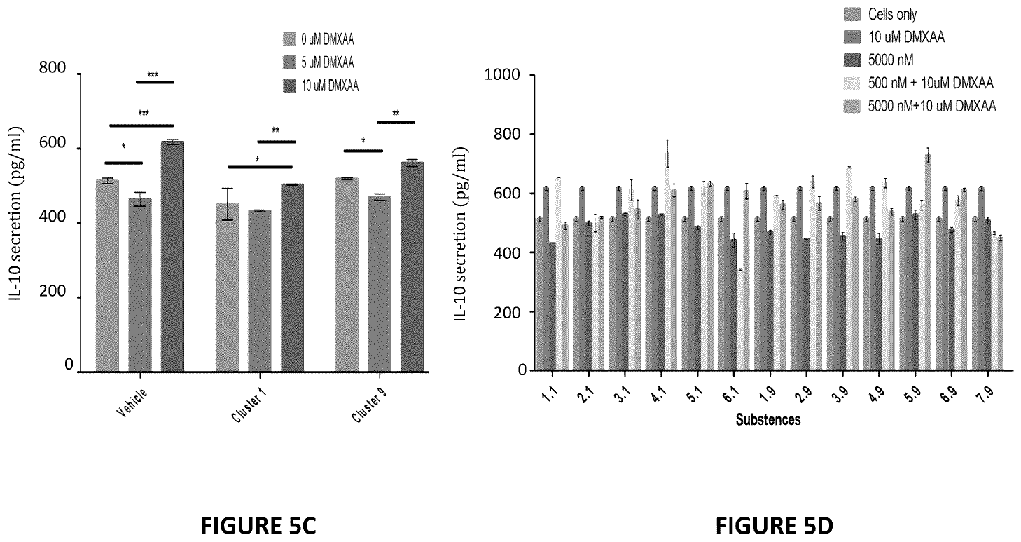

It was hypothesized that STING-binding compounds can serve as biased agonists that limit inflammation by promoting IL-10, but not TNFα, production. To use a more physiological setting, macrophages were isolated from zymosan A-induced peritonitis and treated with the compounds alone (5,000 nM) or compounds (50-5,000 nM) with DMXAA, or DMXAA alone. It was found that compound 7.9 was again the best inhibitor of both TNFα and IL-10, while two other compounds (5.9 and 6.1, at 5000 nM) enhanced TNFα and IL-10 induced by DMXAA (FIG. 5). Compound 3.1 enhanced TNFα only, while compound 4.1 enhanced IL-10 at 500 but not at 5,000 nM. Interestingly, all compounds increased TNFα at 5,000 nM when treated alone, but not to the same extent as DMXAA (FIG. 5). Several compounds increased IL-10 (when used alone) but to a very low extent.

All compounds show some type of activity. Compounds 5.9 and 7.9 seem to act as very efficient STING inhibitors (of both TNFα and IL-10) at low concentrations and in various cell types, while compounds 6.1, 4.1, and 2.1 seem to be biased agonists that induce IL-10 at low concentrations and inhibit TNFα at high concentrations. Compound 5.9 acts as a biased agonist in LPS-stimulated splenocytes that enhances both IL-10 and IFN-β production while inhibiting TNFα. Compound 4.1 is probably the most efficient biased agonist with DMXAA stimulation since it induces IL-10 at 5,000 nM in the absence or presence of DMXAA and in various cell types, while it inhibits TNFα.

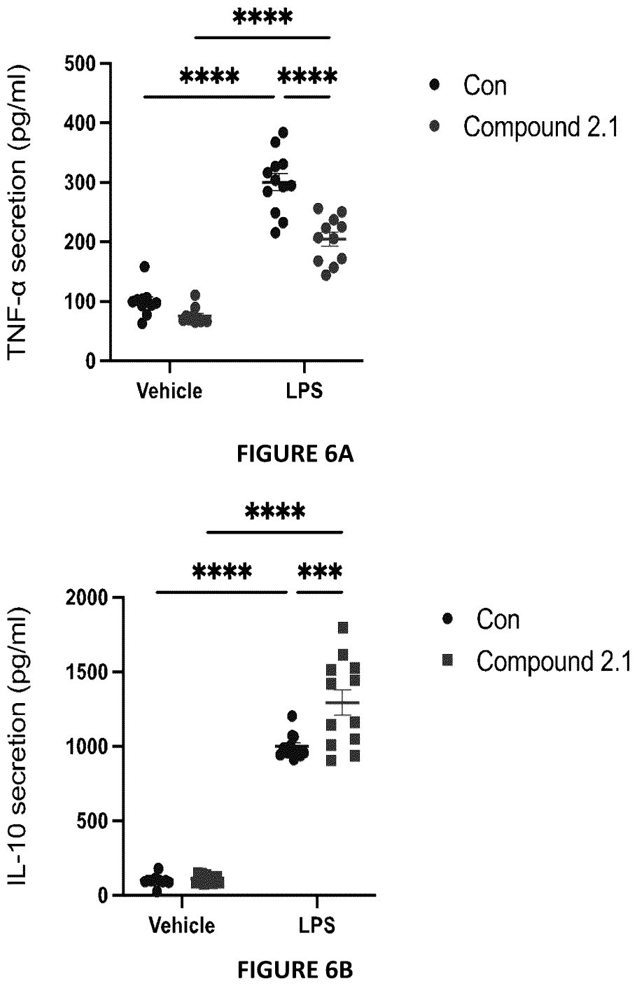

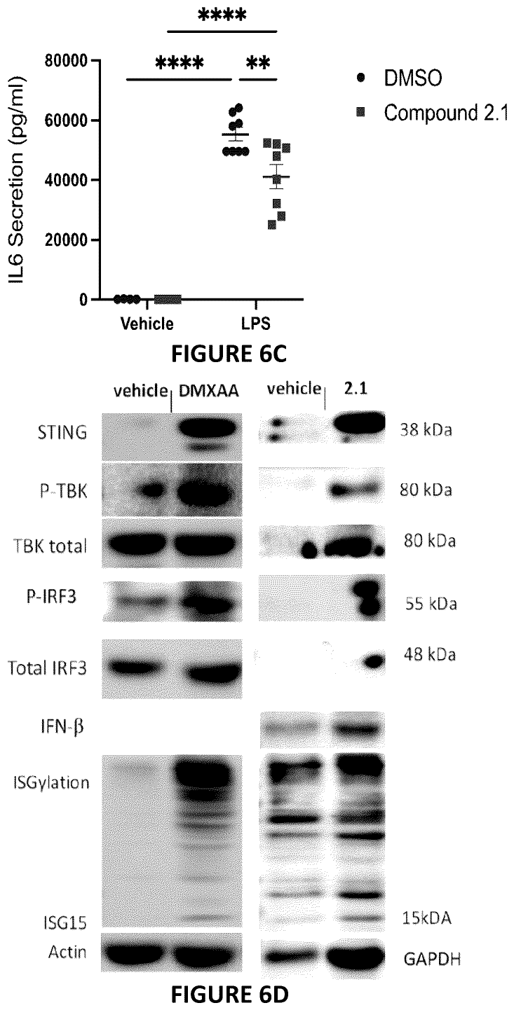

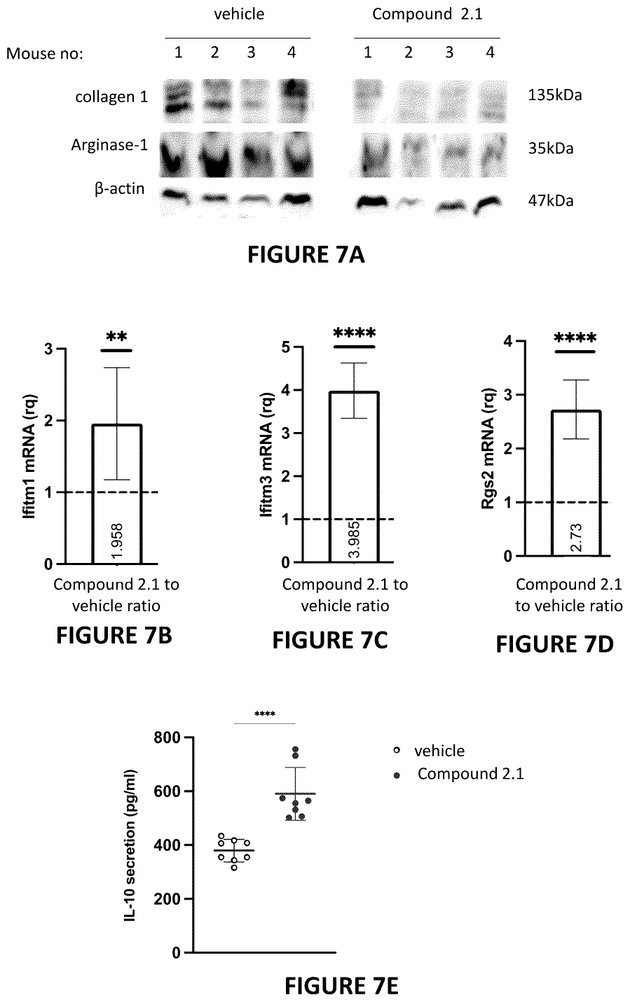

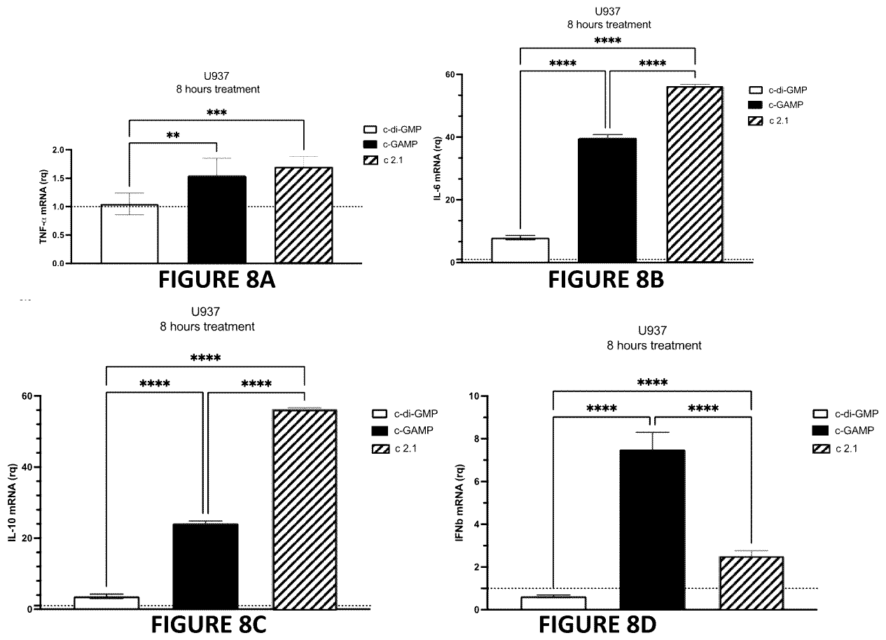

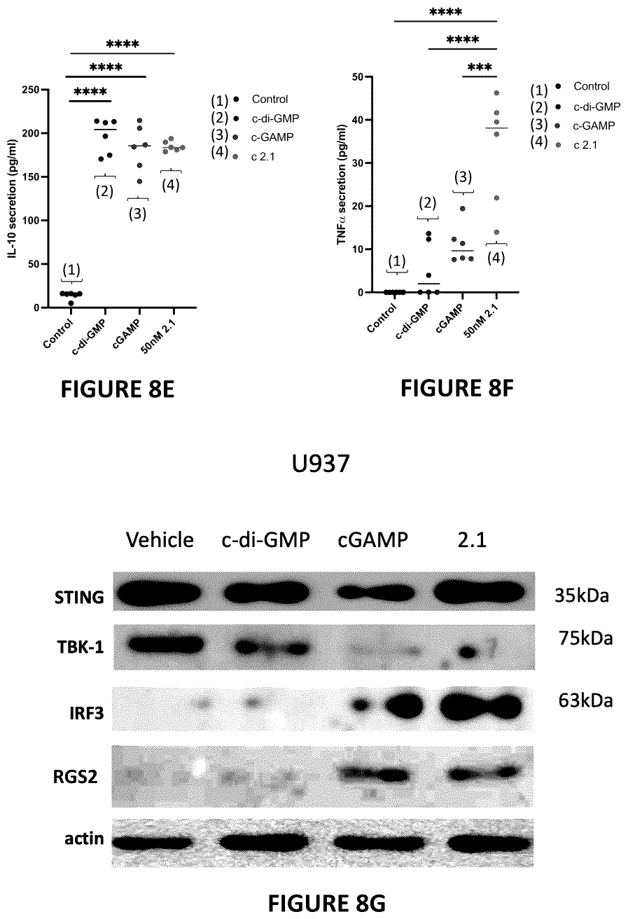

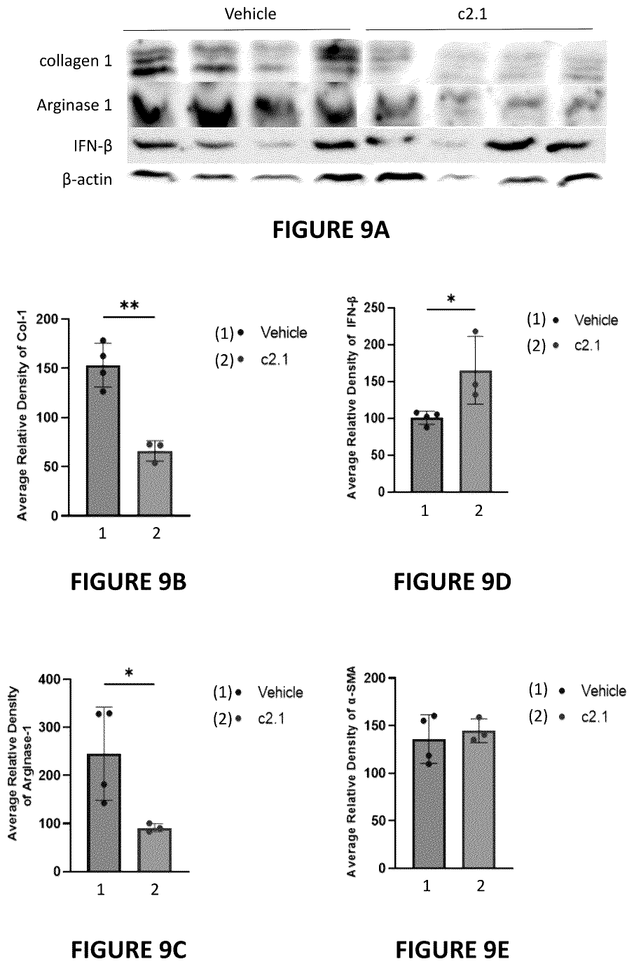

Further, the inventors have demonstrated that compound 2.1 activates the STING-IFN-βaxis in vivo and enhances biased cytokine production by macrophages (FIG. 6), facilitates anti-fibrotic responses during the resolution of liver fibrosis (FIG. 7), and activates cytokine expression and STING signaling in human macrophages in a similar fashion to cGAMP (FIG. 8). Further in the context of the resolution of liver fibrosis, the inventors have shown that simultaneously with the elevated production of IFN-β, treatment with compound 2.1, an anti-fibrotic response was manifested by a reduction in the fibrosis-related factors, Collagen 1 and arginase-1 (FIG. 9).

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A pharmaceutical composition, comprising a pharmaceutically acceptable carrier and at least one stimulator of IFN genes (STING) inhibitor selected from the group consisting of:

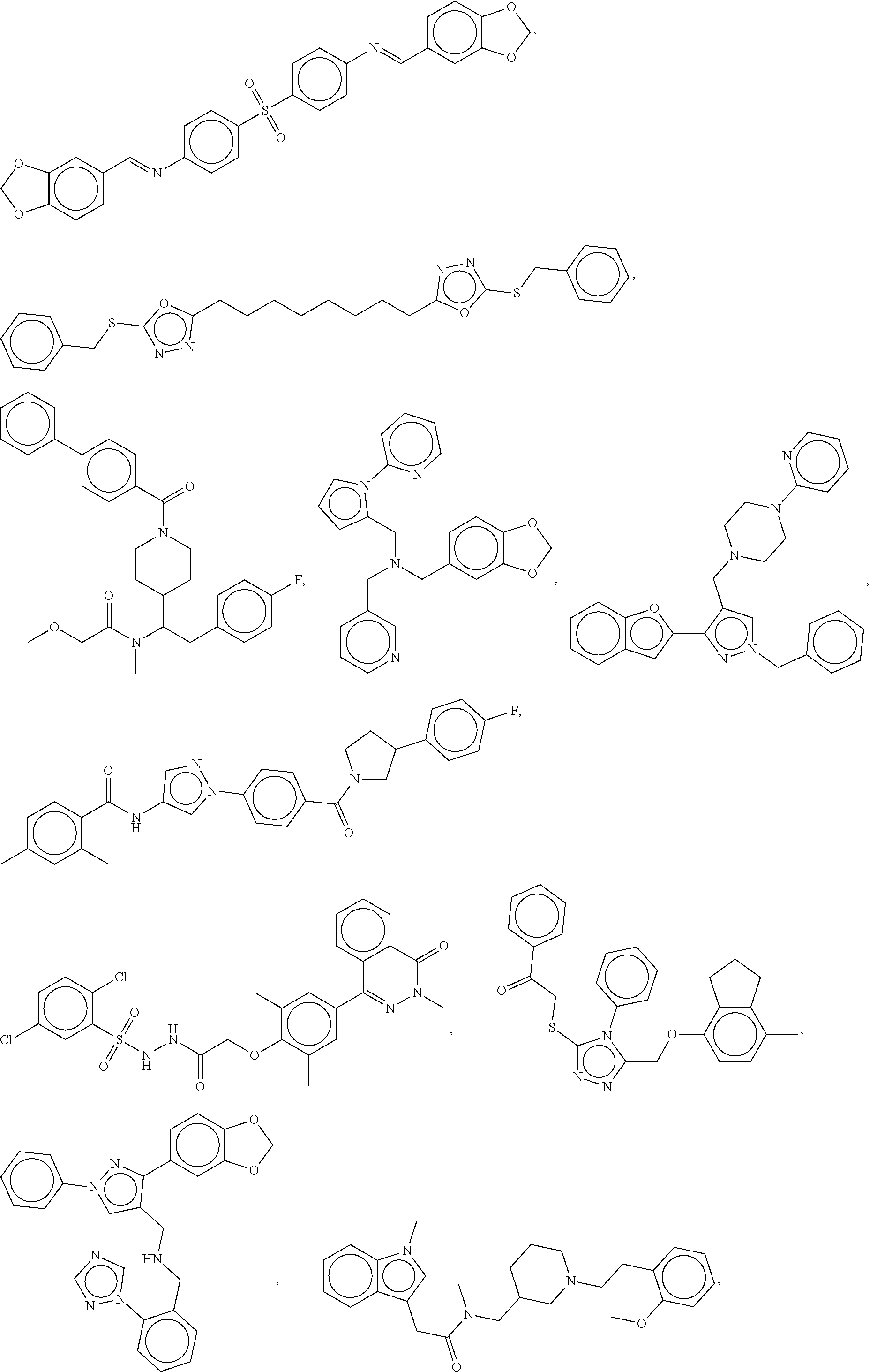

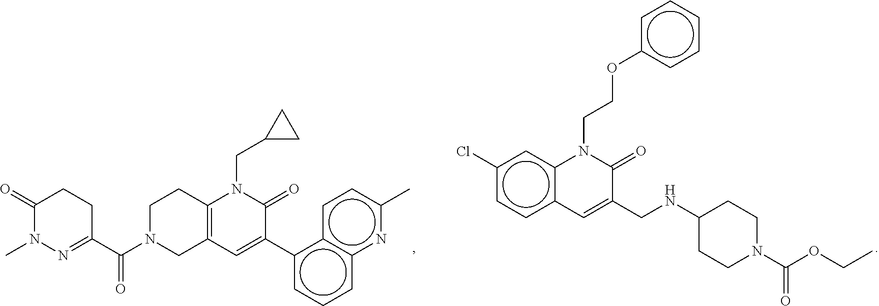

2. A method for inhibiting TNF-alpha secretion from a cell, comprising the step of contacting said cell with an effective amount of a compound selected from the group consisting of: 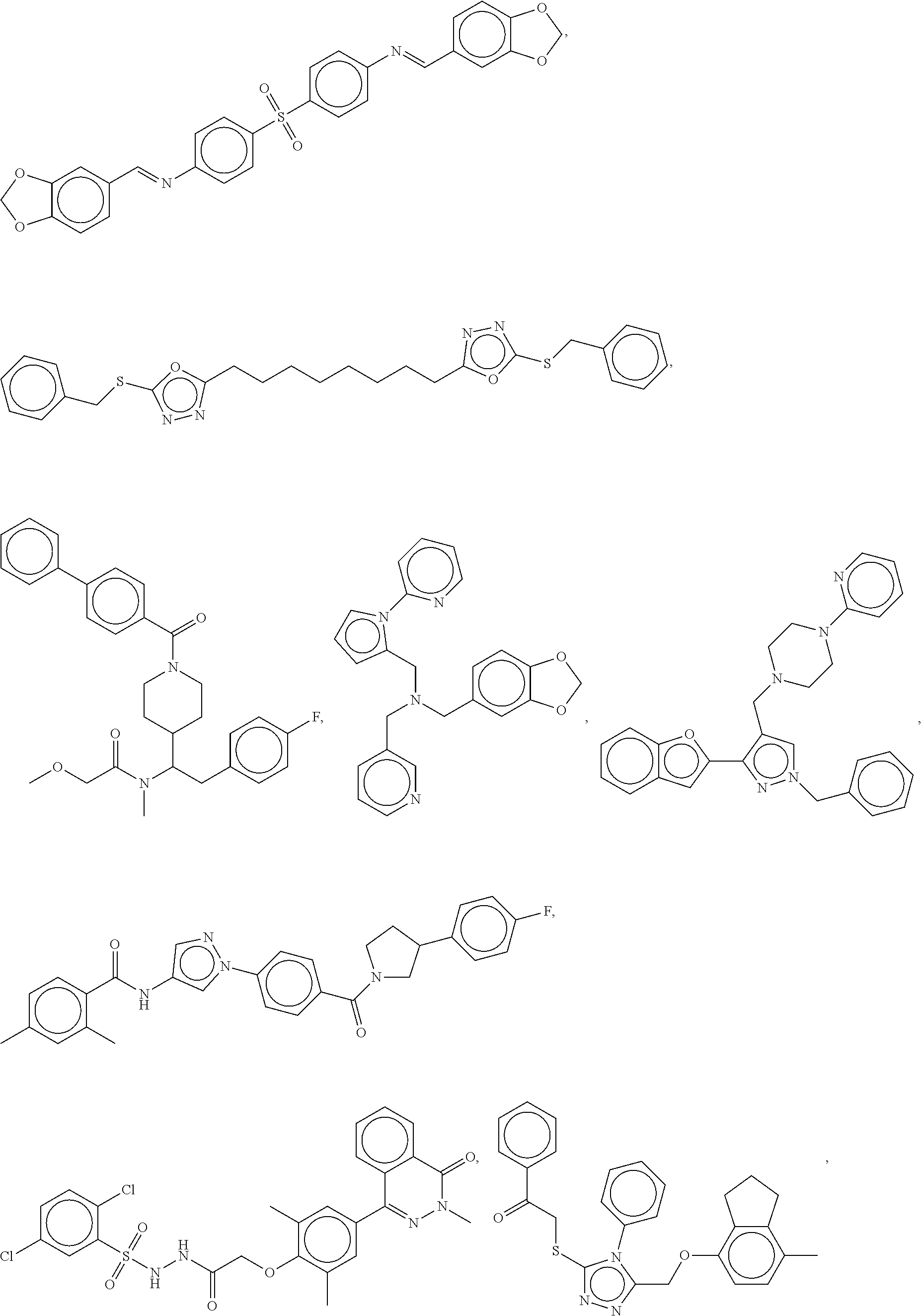 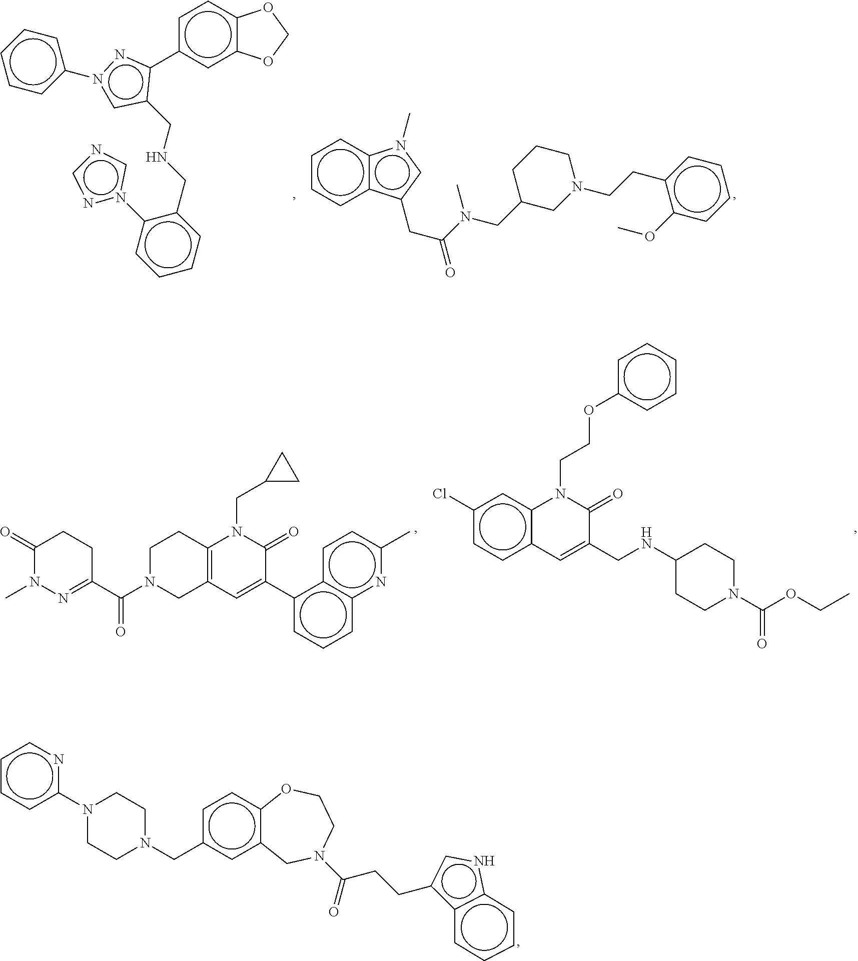
and any combination thereof, thereby inhibiting TNF-alpha secretion from said cell.

3. The method of claim 2, wherein said compound is:

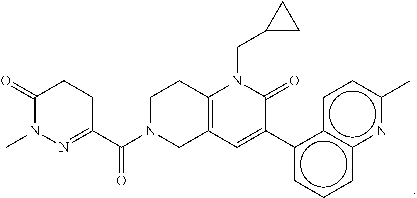

4. The method of claim 2, wherein said compound induces IL-10 secretion, IFN-beta secretion, or both, from said cell.

5. The method of claim 2, wherein said compound inhibits IL-10 secretion, IFN-beta secretion, or both, from said cell.

6. The method of claim 2, wherein said cell is a lymphocyte, a dendritic cell, or a macrophage.

7. The method of claim 2, wherein said cell is an activated inflammatory cell.

8. A method for inhibiting a STING in a cell, comprising the step of contacting said cell with an effective amount of a compound selected from the group consisting of: 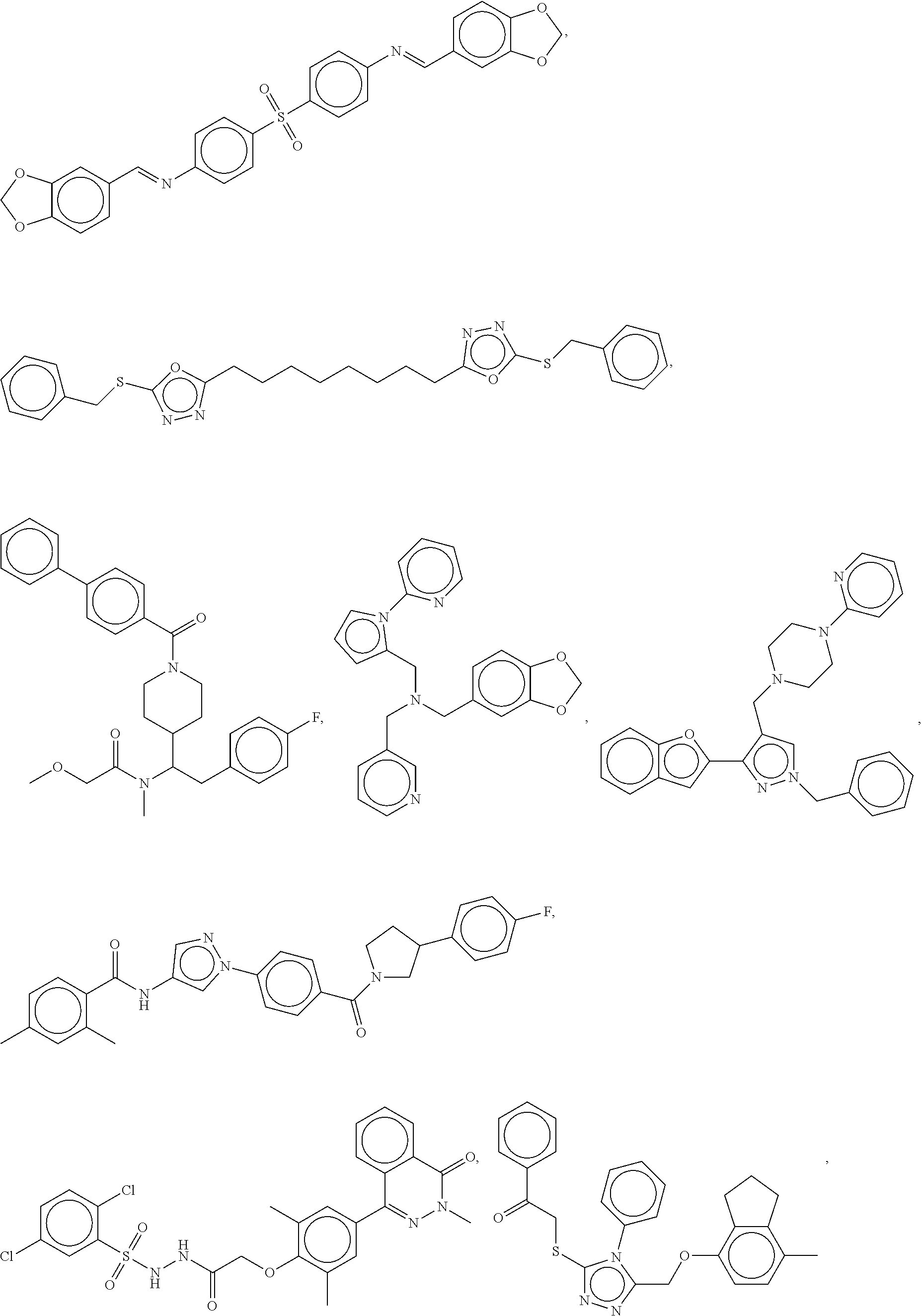 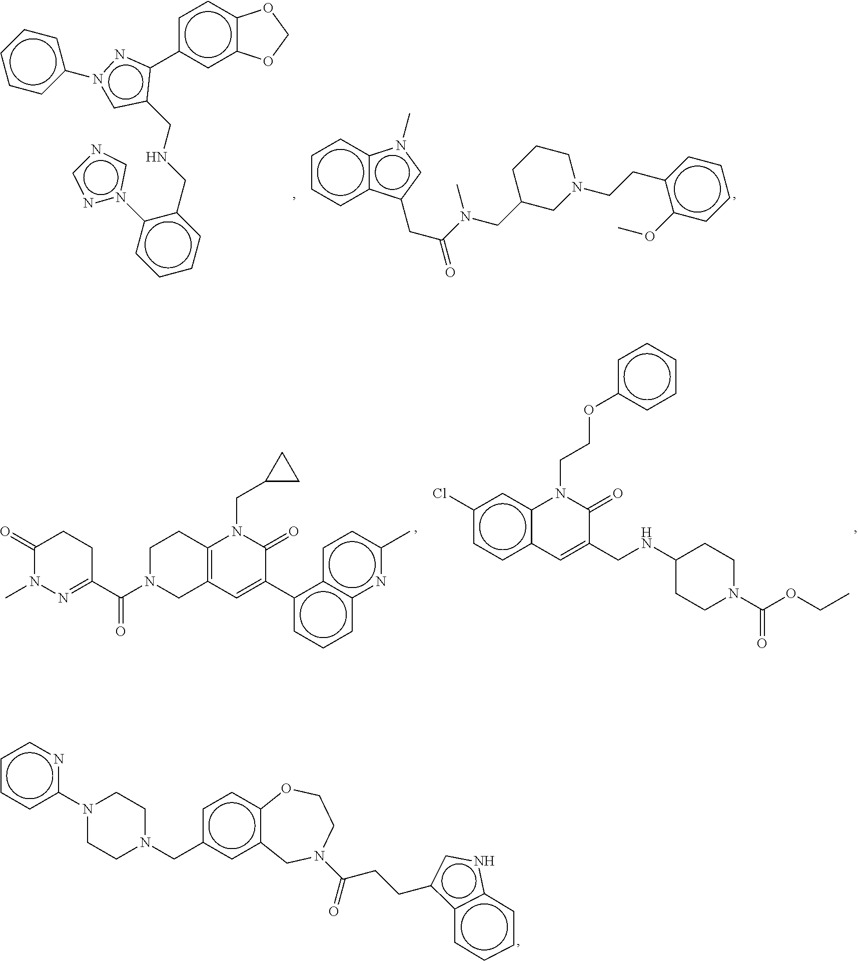
and any combination thereof, thereby inhibiting said STING in said cell.

9. The method of claim 8, wherein said compound is: 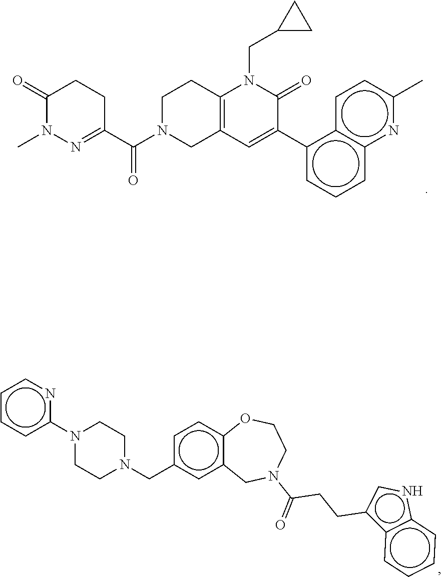
or a combination thereof.

10. A method for treating a fibrotic disease in a subject in need thereof, the method comprising administering to said subject a therapeutically effective amount of the pharmaceutical composition of claim 1, thereby treating a fibrotic disease in the subject.

11. The method of claim 10, wherein said fibrotic disease comprises liver fibrosis, skin fibrosis, lung fibrosis, kidney fibrosis, heart fibrosis, or any combination thereof.

12. The method of claim 10, wherein said fibrotic disease is liver fibrosis.

13. The method of claim 10, wherein said treating comprises: reducing production of Collagen 1, and of Arginase-1, increasing production of IFN-β, or any combination thereof, in the liver of said subject.

14. The method of claim 10, wherein said at least one STING inhibitor is: [chemical structure]
